SEQUENCE LISTING (25 sequences)

SEQ ID NO: 1 - protein, 13 aa, synthetic construct
AHIVMVDAYKPTK

SEQ ID NO: 2 - protein, 93 aa, synthetic construct
MDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 3 - protein, 15 aa, synthetic construct
GGGGSGGGGSGGGGS

SEQ ID NO: 4 - protein, 15 aa, synthetic construct
EAAAKEAAAKEAAAK

SEQ ID NO: 5 - protein, 19 aa, synthetic construct
GGSGGSGSEKAAKAEEAAR

SEQ ID NO: 6 - protein, 9 aa, synthetic construct
GGSGGSGGS

SEQ ID NO: 7 - protein, 12 aa, synthetic construct
GGSGGSGGSGGS

SEQ ID NO: 8 - protein, 6 aa, synthetic construct
SGGSGG

SEQ ID NO: 9 - protein, 9 aa, synthetic construct
GSGGSGGSG

SEQ ID NO: 10 - protein, 19 aa, synthetic construct
MGWSLILLFLVAVATRVLS

SEQ ID NO: 11 - protein, 19 aa, synthetic construct
MEWSWVFLFFLSVTTGVHS

SEQ ID NO: 12 - protein, 19 aa, synthetic construct
MEFGLSWVFLVAIIKGVQC

SEQ ID NO: 13 - protein, 544 aa, synthetic construct
MGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGG

SEQ ID NO: 14 - protein, 514 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGG

SEQ ID NO: 15 - protein, 548 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGGEAAAKEAAAKEAAAKAHIVMVDAYKPTKHHHHHH

SEQ ID NO: 16 - protein, 545 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGGGGGSGGGSGGGSAHIVMVDAYKPTKHHHHHH

SEQ ID NO: 17 - protein, 205 aa, synthetic construct
MKMEELFKKHKIVAVLRANSVEEAKKKALAVFLGGVHLIEITFTVPDADTVIKELSFLKEMGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 18 - protein, 310 aa, synthetic construct
MDSATHIKFSKRDEDGKELAGATMELRDSSGKTISTWISDGQVKDFYLYPGKYTFVETAAPDGYEVATAITFTVNEQGQVTVNGKATKGDAHIGGSGGSGGSGGSMKMEELFKKHKIVAVLRANSVEEAKKKALAVFLGGVHLIEITFTVPDADTVIKELSFLKEMGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 19 - protein, 205 aa, synthetic construct
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 20 - protein, 172 aa, synthetic construct
MNQHSHKDHETVRIAVVRARWHAEIVDACVSAFERRITINAAMRDIGGDRFAVDVFDVPGAYEIPLHARTLAETGRYGAVLGTAFVVNGGIYRHEFVASAVINGMMNVQLNTGVPVLSAVLTPHNYDKSKAHTLLFLALFAVKGMEAARACVEILAAREKIAAGSGHHHHHH

SEQ ID NO: 21 - protein, 765 aa, synthetic construct
MEFGLSWVFLVAIIKGVQCSVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGGGGSGGSGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTEGGSHHHHHH

SEQ ID NO: 22 - protein, 163 aa, synthetic construct
DIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 23 - protein, 271 aa, synthetic construct
MHHHHHHGGSDSATHIKFSKRDEDGKELAGATMELRDSSGKTISTWISDGQVKDFYLYPGKYTFVETAAPDGYEVATAITFTVNEQGQVTVNGKATKGDAHISGGSGGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 24 - protein, 130 aa, synthetic construct
ANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 25 - protein, 244 aa, synthetic construct
MGSSHHHHHHGSGDSATHIKFSKRDEDGKELAGATMELRDSSGKTISTWISDGQVKDFYLYPGKYTFVETAAPDGYEVATAITFTVNEQGQVTVNGKATKGDAHIGSGGSGGSGANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

(19) United States
(12) Patent Application Publication
(10) Pub. No.: US 2026/0232794 A1
(43) Pub. Date: Aug. 13, 2026

(54) Immune Composition Product for Preventing or Treating Varicella Zoster Virus-Related Diseases and Preparation Method Therefor

(71) Applicants: YANTAI PATRONUS BIOTECH CO., LTD., Yantai (CN); GUANGZHOU PATRONUS BIOTECH CO., LTD., Guangzhou (CN)

(72) Inventors: Jing JIN, Guangdong (CN); Yuanyuan LI, Guangdong (CN); Yu ZHOU, Guangdong (CN)

(21) Appl. No.: 19/534,319

(22) Filed: Feb. 9, 2026

Related U.S. Application Data
(63) Continuation of application No. PCT/CN2023/126034, filed on Oct. 23, 2023.

(51) Int. Cl. A61K 39/25; A61K 39/00; A61P 37/04

(52) U.S. Cl. CPC A61K 39/25; A61P 37/04; A61K 2039/55505; A61K 2039/55511; A61K 2039/55555; A61K 2039/627

(57) ABSTRACT

Provided is a varicella-zoster vaccine. Specifically, the vaccine comprises an immune composition, and the immune composition comprises an antigenic component and a particle protein component. The particle protein component comprises a nanoparticle protein, and the antigenic component and the particle protein component covalently bind to each other by means of a binding peptide 1 and a binding peptide 2 to form an immunogenic complex. Provided is a preparation method for the varicella-zoster vaccine.

### CLAIM OF PRIORITY

This application is a continuation application of International Application No. PCT/CN2023/126034, filed on Oct. 23, 2023. The entire contents of the foregoing application are incorporated herein by reference in their entireties.

### SEQUENCE LISTING

This application contains a Sequence Listing that has been submitted electronically as an XML file named “54821-0003001_SL_ST26.xml.” The XML filed, created on Apr. 8, 2026, is 27,406 bytes in size. The material in the XML file is hereby incorporated by reference in its entirety.

### TECHNICAL FIELD

The present disclosure relates to the technical field of bio-pharmaceuticals, and in particular, to an immune composition product for preventing or treating varicella-zoster virus (VZV)-related diseases and a production and preparation method therefor.

### BACKGROUND

Herpes zoster is an erythematous vesicular skin rash distributed along the nerve segments and caused by the reactivation of the varicella-zoster virus (VZV), which causes chickenpox after the primary infection of the human body and subsequently remains latent in spinal dorsal horn neurons before being reactivated by stimuli. It is more commonly observed in elderly individuals, those with immunodeficiencies, or patients using immunosuppressive agents. Its symptoms and lingering postherpetic neuralgia cause significant distress for the patients. So far, there has been no effective therapeutic drug for the disease, and vaccination has become the only way to prevent and treat the disease.

For most people carrying VZV, the virus is latent in neurons. After different incubation periods, VZV can be released and infect other cells. The main protection mechanism of the herpes zoster vaccine is as follows: By inducing a cellular immune response, the virus is inhibited from being activated in neurons and diffused to cells through nerves, and the antibodies generated by humoral immunity also have a certain protective effect.

Currently, two herpes zoster vaccines have been developed worldwide, namely the recombinant vaccine Shingrix® (GSK) and the attenuated vaccine Zostavax® (MERCK). Both vaccines cover people aged 50 or more. gE protein is the most abundantly expressed glycoprotein in VZV and exhibits high immunogenicity. The recombinant vaccine product Shingrix® consists of an active ingredient, an AS01B adjuvant system, and other auxiliary materials. The active ingredient is glycoprotein E (gE) of VZV, which is prepared by transfecting Chinese hamster ovary (CHO) cells with a protein-encoding sequence by DNA recombinant technology, expressing a specific antigen, purifying, and lyophilizing. The gE protein for injection is a sterile white powder. The AS01B adjuvant system suspension is a liposome formulation containing two immune enhancing components (3-O-desacyl-4′-monophosphoryl lipid A (MPL) and quillaja saponin QS-21). The suspension for injection (the AS01B adjuvant system) is an opalescent, colorless to pale brown liquid. The decrease in the number of CD4⁺ T cells and the decrease in the immune response caused by aging are key factors for the activation of VZV virus, and the enhancement of the specific T cell immunity is the core competitive power of the herpes zoster vaccine. The adjuvant AS01B contained in Shingrix® (GSK) can effectively and continuously promote the development and differentiation of specific CD4⁺ T cells in people aged 50 or more. Shingrix® (GSK) has a stronger protection effect on herpes zoster and postherpetic neuralgia caused by herpes zoster compared with Zostavax® (MERCK), and the two vaccines both have been approved for use in multiple countries. Shingrix® is the first herpes zoster vaccine to be launched in China, while Zostavax® has not been launched in China.

Although the Shingrix® herpes zoster vaccine using AS01B can increase the protective efficacy from 51% (achieved by Zostavax®) to over 90%, the Shingrix®/AS01B vaccine also has significant drawbacks, including the limited adjuvant production capacity and pronounced side effects of the vaccine caused by the adjuvant. The ability of the AS01B adjuvant to induce inflammatory reaction is very strong. After vaccination, vaccinees often experience symptoms such as systemic muscle and joint pain, fatigue, and fever. Consequently, a significant portion of the eligible population is unwilling to receive the vaccine, and the vaccination rate and compliance of the vaccine are greatly reduced. Therefore, developing a new vaccine with mild side effects to increase the vaccination rate and compliance of the eligible population is of far-reaching clinical significance.

### SUMMARY

The present disclosure provides a varicella-zoster vaccine and a preparation method therefor. The vaccine is a nanoparticle vaccine. The vaccine can avoid the use of an AS01B adjuvant and thereby eliminates or reduces side effects caused by such adjuvants. Meanwhile, compared with the vaccine Shingrix®, the nanoparticle vaccine exhibits both higher T cell immunogenicity and antibody immunogenicity than those of Shingrix®, thereby providing a higher protective efficacy than Shingrix®, and demonstrating far-reaching clinical value. Meanwhile, it also addresses the issue of insufficient technology and supply variety for varicella-zoster vaccines currently, filling the gap where nanoparticle-based varicella-zoster vaccines are almost unavailable.

A nanoparticle vaccine is a vaccine formed based on a nanoparticle protein. The nanoparticle protein is mainly used for antigen display.

The present disclosure provides an immunogenic complex, which comprises a protein formed by a covalent binding reaction between an antigenic component and a particle protein component.

The present disclosure provides an immune composition, which comprises the immunogenic complex disclosed herein and a pharmaceutically acceptable carrier. The immune composition may be in the form of a lyophilized formulation or an injection formulation.

The present disclosure provides a vaccine, which comprises the immune composition disclosed herein and an adjuvant.

The present disclosure provides an immunogenic complex, which comprises:
(1) an antigenic component comprising a varicella-zoster virus (VZV) gE protein or an immunogenic fragment thereof; and
(2) a particle protein component comprising a nanoparticle protein.

The present disclosure provides an immunogenic complex, which comprises:
(1) an antigenic component comprising a varicella-zoster virus (VZV) gE protein or an immunogenic fragment thereof, a linker peptide 1, and a binding peptide 1; and
(2) a particle protein component comprising a nanoparticle protein, a linker peptide 2, and a binding peptide 2;
wherein the antigenic component and the particle protein component are covalently bound to each other via the binding peptide 1 and the binding peptide 2.

The present disclosure provides an immunogenic complex, which comprises:
(1) an antigenic component consisting of a varicella-zoster virus (VZV) gE protein or an immunogenic fragment thereof, a linker
peptide 1, and a binding peptide 1; and
(2) a particle protein component consisting of a nanoparticle protein, a linker peptide 2, and a binding peptide 2;
wherein the antigenic component and the particle protein component are covalently bound to each other via the binding peptide 1 and the binding peptide 2.

In some embodiments, in any one of the immunogenic complexes provided herein, the antigenic component is formed by fusing the VZV gE protein, at its C-terminus, to the binding peptide 1 via the linker peptide 1.

In some embodiments, the “immunogenic fragment” refers to a portion of an oligopeptide, a polypeptide, or a protein that is immunogenic and elicits a protective immune response when administered to a subject.

In some embodiments, in any one of the immunogenic complexes provided herein, the particle protein component is formed by fusing the nanoparticle protein, at its N-terminus, to the binding peptide 2 via the linker peptide 2.

In some embodiments, in any one of the immunogenic complexes provided herein, the antigenic component is as follows from the N-terminus to the C-terminus: the varicella-zoster virus (VZV) gE protein or the immunogenic fragment thereof, the linker peptide 1, and the binding peptide 1; the particle protein component is as follows from the N-terminus to the C-terminus: the binding peptide 2, the linker peptide 2, and the nanoparticle protein; the antigenic component and the particle protein component are covalently bound to each other via the binding peptide 1 and the binding peptide 2, thus forming the immunogenic complex.

In some embodiments, in any one of the immunogenic complexes provided herein, the antigenic component and/or the particle protein component comprise a histidine tag.

The present disclosure provides an immunogenic complex, which comprises:
(1) an antigenic component comprising a varicella-zoster virus (VZV) gE protein or an immunogenic fragment thereof and a linker peptide 1; and
(2) a particle protein component comprising a nanoparticle protein subunit.

In some embodiments, the VZV gE protein is linked to one subunit of the nanoparticle protein to form a fusion protein, which then binds to the other subunit of the nanoparticle protein.

In some embodiments, in any one of the immunogenic complexes provided herein, the particle protein component comprises a nanoparticle protein. Preferably, the nanoparticle protein may be a virus-like particle protein, and the virus-like particle protein is formed from a viral structural protein, preferably from a bacteriophage capsid protein AP205. The particle protein component and the antigenic component may be covalently bound to form a particle structure.

In some embodiments, in any one of the immunogenic complexes provided herein, the nanoparticle protein used may also be selected from an NPM particle, a ferritin particle (Ferritin), an I53-50 particle, and the like.

In some embodiments, in any one of the immunogenic complexes provided herein, the nanoparticle protein I53-50 particle used consists of two subunits I53-50A and I53-50B.

In some embodiments, in any one of the immunogenic complexes provided herein, the binding peptide 1 comprises the amino acid sequence set forth in SEQ ID NO: 1.

In some embodiments, in any one of the immunogenic complexes provided herein, the binding peptide 2 comprises the amino acid sequence set forth in SEQ ID NO: 2.

In some embodiments, in any one of the immunogenic complexes provided herein, the linker peptide 1 comprises the amino acid sequence of (GGGGS)ₙ or (EAAAK)ₙ, wherein n may be an integer greater than 0 and less than or equal to 5. In some embodiments, in any one of the immunogenic complexes provided herein, the linker peptide 1 is preferably (GGGGS)₃ (SEQ ID NO: 3), (EAAAK)₃ (SEQ ID NO: 4), or GGSGGSGSEKAAKAEEAAR (SEQ ID NO: 5).

In some embodiments, in any one of the immunogenic complexes provided herein, the linker peptide 2 comprises the amino acid sequence of (GGS)ₙ, (SGGSGG)ₙ, or (GSGGSGGSG)ₙ, wherein n may be an integer greater than 0 and less than or equal to 10. In some embodiments, in any one of the immunogenic complexes provided herein, the linker peptide 2 is preferably GGSGGSGGS (SEQ ID NO: 6), GGSGGSGGSGGS (SEQ ID NO: 7), SGGSGG (SEQ ID NO: 8), or GSGGSGGSG (SEQ ID NO: 9).

Preferably, the varicella-zoster virus (VZV) gE protein disclosed herein is expressed using a signal peptide with the amino acid sequence set forth in SEQ ID NO: 10, SEQ ID NO: 11, or SEQ ID NO: 12; more preferably, the sequence of the signal peptide is SEQ ID NO: 12.

Specifically, since the sequence of the first 544 amino acids of the VZV gE protein falls outside the transmembrane domain and is antigenic, the VZV gE protein disclosed herein is designed based on the sequence of amino acids at positions 1-544 of the VZV gE (the amino acids 1-544 of VZV gE are set forth in SEQ ID NO:13), wherein amino acids at positions 1-30 are the inherent natural secretory signal peptide of VZV gE, and amino acids at positions 31-544 of VZV gE are the amino acid sequence without signal peptide (amino acid sequence set forth in SEQ ID NO:14). In the present disclosure, the sequence of positions 31-544 of VZV gE is used, or non-natural signal peptides with other sequence structures are used to replace the above natural signal peptides to increase the protein expression level. The above sequence without a signal peptide or a sequence formed by linking an additional signal peptide with a non-natural structure to the amino acids at positions 31-544 of VZV gE is used, VZV gE is linked to a binding peptide 1 (the binding peptide 1 is named “4T”) at the C-terminus via a specific linker peptide 1 (linker 1), and meanwhile, a histidine (e.g., 6His) purification tag may be added to the C-terminus of the fusion protein. The encoding gene encoding the above fusion protein is inserted into a eukaryotic cell expression vector (e.g., pcDNA3.4) for expression in CHO cells to obtain a fusion protein formed by VZV gE-binding peptide 1. The antigenic component is subjected to nickel column affinity chromatography, size exclusion chromatography, and the like to obtain a high-purity protein, and the antigenic component is VZV gE-4T.

In some embodiments, in any one of the immunogenic complexes provided herein, the varicella-zoster virus (VZV) gE protein comprises the amino acid sequence set forth in SEQ ID NO: 14.

In some embodiments, in any one of the above immunogenic complexes provided herein, the particle protein component is a fusion protein formed by fusing the nanoparticle protein, at its N-terminus, to a binding peptide 2 via a linker peptide 2; preferably, the nanoparticle protein is NPM, AP205 capsid protein 3 (AP205), or Ferritin protein. Specifically, in some optional embodiments, the binding peptide 2 (the binding peptide 2 is named “4C”) is linked to the encoding gene of the nanoparticle protein via the linker peptide 2, and the construct is inserted into a prokaryotic expression vector (such as pET-28a(+) or pET-30a(+)) for expression in E. coli cells to obtain a fusion protein of the binding peptide 2 and the nanoparticle protein. The fusion protein may be purified by chromatography (e.g., anion exchange chromatography and hydrophobic chromatography) to obtain a product. The nanoparticle protein is preferably NPM, AP205, or Ferritin; the formed particle protein components are named NPM-4C, AP205-4C, and Ferritin-4C.

Specifically, in some optional embodiments, any one of the above antigenic components is subjected to a conjugate binding reaction with the particle protein component under suitable reaction conditions, and the conjugation is achieved by the formation of a covalent bond between the binding peptide 1 of the antigenic component and the binding peptide 2 of the particle protein component, thereby forming the immunogenic complex. Different immunogenic complexes can be formed using different nanoparticle proteins, and these immunogenic complexes are separately named VZV gE-NPM, VZV gE-AP205, or VZV gE-Ferritin.

Preferably, in any one of the immunogenic complexes provided herein, the antigenic component is expressed using a signal peptide MEFGLSWVFLVAIIKGVQC (SEQ ID NO: 12) and comprises a VZV gE protein (SEQ ID NO: 14), a linker peptide 1 (EAAAK)₃ (SEQ ID NO: 4), a binding peptide 1 (SEQ ID NO: 1), and a histidine tag; more preferably, the sequence of the antigenic component is set forth in SEQ ID NO: 15.

Preferably, in any one of the immunogenic complexes provided herein, the antigenic component is expressed using a signal peptide MEFGLSWVFLVAIIKGVQC (SEQ ID NO: 12) and comprises a VZV gE protein (SEQ ID NO: 14), a linker peptide 1 (GGGGS)₃ (SEQ ID NO: 3), a binding peptide 1 (SEQ ID NO: 1), and a histidine tag; more preferably, the sequence of the antigenic component VZV gE-4T is set forth in SEQ ID NO: 16.

In some embodiments, the present disclosure provides an immunogenic complex, which comprises:
(1) an antigenic component comprising a varicella-zoster virus (VZV) gE protein, a linker peptide 1, and a binding peptide 1; and
(2) a particle protein component comprising a nanoparticle protein, a linker peptide 2, and a binding peptide 2.

The linker peptide 1 is any linker peptide commonly used in the art, including but not limited to the amino acid sequence of (GGGGS)ₙ or (EAAAK)ₙ, where n may be an integer greater than 0 and less than or equal to 5, and the linker peptide 1 is preferably SEQ ID NO: 3 or SEQ ID NO: 4; the linker peptide 2 is any linker peptide commonly used in the art, including but not limited to the amino acid sequence of (GGS)ₙ, (SGG)ₙ, or (GSGGSGGSG)ₙ, wherein n may be an integer greater than 0 and less than or equal to 10, and the linker peptide 2 is preferably SEQ ID NO: 6, SEQ ID NO: 7, SEQ ID NO: 8, or SEQ ID NO: 9; the nanoparticle protein is NPM, AP205 or Ferritin.

In some embodiments, in any one of the immunogenic complexes provided herein, the nanoparticle protein NPM comprises the amino acid sequence set forth in SEQ ID NO: 17.

Preferably, in any one of the immunogenic complexes provided herein, the particle protein component comprises NPM-4C, as set forth in SEQ ID NO: 18, which is a fusion protein obtained by linking the binding peptide 2 set forth in SEQ ID NO: 2 to the nanoparticle protein NPM set forth in SEQ ID NO: 17 via the linker peptide 2 set forth in SEQ ID NO: 7.

In some other embodiments, the present disclosure provides an immunogenic complex, which comprises:
(1) an antigenic component comprising a varicella-zoster virus (VZV) gE protein and a linker peptide 1, wherein the linker peptide 1 comprises the amino acid sequence set forth in SEQ ID NO: 5; and
(2) a particle protein component comprising a nanoparticle protein subunit, wherein preferably, the nanoparticle protein subunit is an I53-50A subunit and/or an I53-50B subunit.

In some embodiments, in any one of the immunogenic complexes provided herein, the nanoparticle protein I53-50 comprises I53-50A and/or I53-50B subunits; preferably, the I53-50A comprises the amino acid sequence set forth in SEQ ID NO: 19, and the I53-50B comprises the amino acid sequence set forth in SEQ ID NO: 20.

Specifically, in any one of the immunogenic complexes provided herein, the varicella-zoster virus (VZV) gE protein is linked to one subunit of the nanoparticle protein to form a fusion protein, which then binds to the other subunit of the nanoparticle protein. Preferably, the subunit of the nanoparticle protein is I53-50A or I53-50B. Further, in some optional embodiments, the VZV gE protein in the antigenic component binds to the nanoparticle protein I53-50A subunit via the linker peptide 1 at the C-terminus to form a VZV gE-I53-50A fusion protein, and the fusion protein then binds to the nanoparticle protein I53-50B subunit.

As described above, when I53-50 is selected as the nanoparticle protein, I53-50 comprises two subunits I53-50A and I53-50B. The extracellular region of the above VZV gE protein with or without a specific signal peptide is linked to I53-50A via the linker peptide 1, and a histidine (e.g., 6H) purification tag may be added to the C-terminus. The encoding gene encoding the above fusion protein is inserted into a eukaryotic cell expression vector (e.g., pcDNA3.4) for expression and purification in CHO cells, and the resulting fusion protein is named VZV gE-I53-50A; meanwhile, a histidine (e.g., 6H) purification tag may be added to the C-terminus of I53-50B, and the gene encoding the above protein is inserted into a prokaryotic cell expression vector (e.g., pET-30a(+)) for expression and purification in E. coli cells, and the resulting protein is named I53-50B. Then, the VZV gE-I53-50A is subjected to a covalent binding reaction with the I53-50B under suitable reaction conditions to form a varicella-zoster nanoparticle, which is named VZV gE-I53-50.

Preferably, any one of the immunogenic complexes provided herein comprises VZV gE-I53-50A (using the signal peptide set forth in SEQ ID NO: 12 for protein expression and comprising a fusion protein obtained by linking the VZV gE protein set forth in SEQ ID NO: 14 to I53-50A set forth in SEQ ID NO: 19 via the linker peptide 1), as set forth in SEQ ID NO: 21.

In some embodiments, in any one of the immunogenic complexes provided herein, the nanoparticle protein Ferritin comprises the amino acid sequence set forth in SEQ ID NO: 22.

Preferably, in any one of the immunogenic complexes provided herein, the particle protein component comprises Ferritin-4C (a fusion protein formed by linking the binding peptide 2 set forth in SEQ ID NO: 2 to the nanoparticle protein Ferritin set forth in SEQ ID NO: 22 via the linker peptide 2 set forth in SEQ ID NO: 8), as set forth in SEQ ID NO: 23.

In some embodiments, in any one of the immunogenic complexes provided herein, the nanoparticle protein AP205 comprises the amino acid sequence set forth in SEQ ID NO: 24.

Preferably, in any one of the immunogenic complexes provided herein, the particle protein component comprises AP205-4C (a fusion protein formed by linking the binding peptide 2 set forth in SEQ ID NO: 2 to the nanoparticle protein AP205 set forth in SEQ ID NO: 24 via the linker peptide 2 set forth in SEQ ID NO: 9), as set forth in SEQ ID NO: 25.

In some embodiments, the present disclosure provides an immunogenic complex, which comprises any one or more of the following (1)-(7):
(1) the amino acid sequence of the varicella-zoster virus (VZV) gE protein is set forth in SEQ ID NO: 14;
(2) the amino acid sequence of the linker peptide 1 is set forth in SEQ ID NO: 3, SEQ ID NO: 4, or SEQ ID NO: 5;
(3) the amino acid sequence of the binding peptide 1 is set forth in SEQ ID NO: 1; (4) the nanoparticle protein is selected from NPM, AP205, or Ferritin;
(5) the nanoparticle protein subunit is selected from I53-50A and/or I53-50B;
(6) the linker peptide 2 comprises the amino acid sequence of (GGS)n, (SGGSGG)n, or (GSGGSGGSG)n, wherein n may be an integer greater than 0 and less than or equal to 10; and
(7) the amino acid sequence of the binding peptide 2 is set forth in SEQ ID NO: 2.

In some embodiments, the present disclosure provides an immunogenic complex, which comprises any one or more of the following (1)-(3):
(1) the amino acid sequence of the NPM is set forth in SEQ ID NO: 17, the amino acid sequence of the Ferritin is set forth in SEQ ID NO: 22, and the amino acid sequence of the AP205 is set forth in SEQ ID NO: 24;
(2) the amino acid sequence of the I53-50A is set forth in SEQ ID NO: 19, and the amino acid sequence of the I53-50B is set forth in SEQ ID NO: 20; and
(3) the amino acid sequence of the linker peptide 2 is set forth in SEQ ID NO: 6, SEQ ID NO: 7, SEQ ID NO: 8, or SEQ ID NO: 9.

In some embodiments, the present disclosure provides an immunogenic complex, which comprises any one or more of the following (1)-(6):
(1) the amino acid sequence of the varicella-zoster virus (VZV) gE protein is set forth in SEQ ID NO: 14;
(2) the amino acid sequence of the linker peptide 1 is set forth in SEQ ID NO: 3 or SEQ ID NO: 4;
(3) the amino acid sequence of the binding peptide 1 is set forth in SEQ ID NO: 1;
(4) the nanoparticle protein is selected from NPM, AP205, or Ferritin, wherein the amino acid sequence of the NPM is set forth in SEQ ID NO: 17, the amino acid sequence of the Ferritin is set forth in SEQ ID NO: 22, and the amino acid sequence of the AP205 is set forth in SEQ ID NO: 24;
(5) the amino acid sequence of the linker peptide 2 is set forth in SEQ ID NO: 7, SEQ ID NO: 8, or SEQ ID NO: 9; and
(6) the amino acid sequence of the binding peptide 2 is set forth in SEQ ID NO: 2.

In some embodiments, the present disclosure provides an immunogenic complex, which comprises any one or more of the following (1)-(6):
(1) the amino acid sequence of the varicella-zoster virus (VZV) gE protein is set forth in SEQ ID NO: 14;
(2) the amino acid sequence of the linker peptide 1 is set forth in SEQ ID NO: 4;
(3) the amino acid sequence of the binding peptide 1 is set forth in SEQ ID NO: 1;
(4) the nanoparticle protein is NPM, and the amino acid sequence of the NPM is set forth in SEQ ID NO: 17;
(5) the amino acid sequence of the linker peptide 2 is set forth in SEQ ID NO: 7; and
(6) the amino acid sequence of the binding peptide 2 is set forth in SEQ ID NO: 2.

In some embodiments, the present disclosure provides an immunogenic complex, wherein the varicella-zoster virus (VZV) gE protein is expressed using a signal peptide with an amino acid sequence set forth in any one of SEQ ID NOs: 10-12, and the antigenic component and/or the particle protein component comprise a histidine tag.

In some embodiments, the present disclosure provides an immunogenic complex, which consists of an antigenic component and a particle protein component, wherein the amino acid sequence of the antigenic component is set forth in SEQ ID NO: 15, and the amino acid sequence of the particle protein component is set forth in SEQ ID NO: 18.

Further, the present disclosure also provides a preparation method for any one of the above immunogenic complexes, which comprises the following steps:
(1) ligating an encoding gene of the antigenic component and an encoding gene of the particle protein component into expression vectors respectively to construct recombinant expression plasmids and expression host strains, and then expressing and purifying target proteins; and
(2) co-incubating the antigenic component and the particle protein component obtained in step (1) to obtain the immunogenic complex.

The present disclosure provides a preparation method for an immunogenic complex for preventing or treating varicella-zoster virus-related diseases:
(1) ligating an encoding gene of the varicella-zoster virus antigenic component and an encoding gene of the particle protein component into expression vectors respectively to construct recombinant expression plasmids;
(2) constructing recombinant strains capable of expressing the varicella-zoster virus antigenic component and the particle protein component in the host cells;
(3) expressing fusion proteins using the recombinant strains and purifying the fusion proteins; and
(4) co-incubating the antigenic component and the particle protein component described above to perform a conjugate binding reaction, thus obtaining the immunogenic complex.

Preferably, the immunogenic complex obtained in step (4) above is purified to obtain a vaccine drug substance.

Preferably, in step (1) of the preparation method for an immunogenic complex for preventing or treating varicella-zoster virus-related diseases, the plasmid expressing the varicella-zoster virus antigenic component may be selected from pcDNA3.4, and the plasmid expressing the particle protein component may be selected from pET-28a(+) and pET-30a(+).

In step (2) of the preparation method for an immunogenic complex for preventing or treating varicella-zoster virus-related diseases disclosed herein, the host cell expressing the varicella-zoster virus antigen is CHO, and the host cell expressing the particle protein component vector is E. coli.

The present disclosure provides an immunogenic complex for preventing or treating varicella-zoster virus-related diseases, wherein the antigenic component of the immunogenic complex comprises the fusion protein formed by the above VZV gE-binding peptide 1.

In the immunogenic complex for preventing or treating varicella-zoster virus-related diseases disclosed herein, the binding ratio of VZV gE-4T to NPM-4C is 6:1, and the binding conditions are as follows: pH 7.4, 0.1 M Tris-HCl, 25% (w/v) Sucrose, reaction at 22° C. for 48 h; the binding ratio of VZV gE-I53-50A to I53-50B is 1:3, and the binding conditions are as follows: pH 7.4, 20 mM Tris-HCl, 150 mM NaCl, reaction at 25° C. for 2 h; the binding ratio of VZV gE-4T to AP205-4C is 2:1, and the binding conditions are as follows: pH 6.2, 40 mM Na₂HPO₄, 25% (w/v) Sucrose, 200 mM sodium citrate (Na₃C₆H₅O₇·2H₂O), reaction at 22° C. for 24 h; the binding ratio of VZV gE-4T to Ferritin-4C is 6:1, and the binding conditions are as follows: pH 7.4, 0.1 M Tris-HCl, 25% (w/v) Sucrose, reaction at 22° C. for 48 h. All endotoxin measurements are below 100 EU/mL, meeting the requirements for large-scale production.

The present disclosure also provides an immune composition, which comprises any one of the above immunogenic complexes and a pharmaceutically acceptable carrier. Preferably, the pharmaceutically acceptable carrier comprises a stabilizer, an excipient, a surfactant, a buffering agent, and a pH regulator. The stabilizer is sucrose and arginine, the excipient is mannitol, the surfactant is Tween 80, the buffering agent is disodium hydrogen phosphate dihydrate and sodium dihydrogen phosphate dihydrate, and the pH regulator is hydrochloric acid.

In some embodiments, the immune composition disclosed herein comprises the immunogenic complex in an amount of 0.25-100 μg/dose, preferably 0.5-50 μg/dose, more preferably 0.5 μg/dose, 1 μg/dose, 2 μg/dose, 3 μg/dose, 4 μg/dose, 5 μg/dose, 10 μg/dose, 15 μg/dose, 20 μg/dose, 25 μg/dose, 30 μg/dose, 35 μg/dose, 40 μg/dose, 45 μg/dose, or 50 μg/dose. The dose for mouse experiment is 1/10 of the human dose.

In some embodiments, the immune composition provided herein is an injection or a lyophilized formulation, preferably a lyophilized formulation.

In some embodiments, the immune composition provided herein is a lyophilized formulation, which comprises a VZV gE-NPM immunogenic complex, a stabilizer, an excipient, a surfactant, a buffering agent, and a pH regulator; preferably, the stabilizer is sucrose and arginine, the excipient is mannitol, the surfactant is Tween 80, the buffering agent is disodium hydrogen phosphate dihydrate and sodium dihydrogen phosphate dihydrate, and the pH regulator is hydrochloric acid.

In some embodiments, the immune composition provided herein is a lyophilized formulation, which comprises a VZV gE-NPM immunogenic complex, sucrose, arginine, mannitol, Tween 80, disodium hydrogen phosphate dihydrate, sodium dihydrogen phosphate dihydrate, and hydrochloric acid. The lyophilized formulation comprises, per unit dose: 0.5-50 μg, preferably 25-50 μg, of VZV gE-NPM; 10-20 mg, preferably 12-15 mg, of sucrose; 10-30 mg, preferably 20-25 mg, of mannitol; 0.1-0.5 mg, preferably 0.2-0.3 mg, of Tween 80; 2-8 mg, preferably 3-5 mg, of arginine; 0.5-1.5 mg, preferably 1-1.2 mg, of disodium hydrogen phosphate dihydrate; 0.5-1 mg, preferably 0.6-0.8 mg, of sodium dihydrogen phosphate dihydrate; and 8.0-9.5 mg, preferably 8.2-9.0 mg, of hydrochloric acid.

In some embodiments, the immune composition provided herein is a lyophilized formulation, which comprises 25 μg or 50 μg of VZV gE-NPM immunogenic complex, 12.5 mg of sucrose, 25 mg of mannitol, 0.25 mg of Tween 80, 4.35 mg of arginine, 1.085 mg of disodium hydrogen phosphate dihydrate, 0.62 mg of sodium dihydrogen phosphate dihydrate, and 8.66 mg of hydrochloric acid.

In some embodiments, the immune composition provided herein is an injection, which comprises a VZV gE-NPM immunogenic complex, a stabilizer, a surfactant, a buffering agent, and a pH regulator; preferably, the stabilizer is sucrose, the surfactant is Tween 80, the buffering agent is disodium hydrogen phosphate dihydrate and sodium dihydrogen phosphate dihydrate, and the pH regulator is hydrochloric acid.

In some embodiments, the immune composition provided herein is an injection, which comprises a VZV gE-NPM immunogenic complex, sucrose, Tween 80, disodium hydrogen phosphate dihydrate, sodium dihydrogen phosphate dihydrate, and hydrochloric acid. The injection comprises, per unit dose: 0.5-50 μg, preferably 25-50 μg, of VZV gE-NPM immunogenic complex; 10-30 mg, preferably 15-25 mg, of sucrose; 0.05-0.5 mg, preferably 0.1-0.3 mg, of Tween 80; 0.2-1 mg, preferably 0.3-0.8 mg, of disodium hydrogen phosphate dihydrate; 0.1-0.5 mg, preferably 0.2-0.4 mg, of sodium dihydrogen phosphate dihydrate; and 0.2-0.5 mg, preferably 0.25-0.35 mg, of hydrochloric acid.

In some embodiments, the immune composition provided herein is an injection, which comprises 25 μg or 50 μg of VZV gE-NPM immunogenic complex, 20 mg of sucrose, 0.125 mg of Tween 80, 0.5425 mg of disodium hydrogen phosphate dihydrate, 0.31 mg of sodium dihydrogen phosphate dihydrate, and 0.3 mg of hydrochloric acid.

The present disclosure further provides a varicella-zoster vaccine, which comprises any one of the above immune compositions and an adjuvant. The adjuvant is selected from at least one of an aluminum salt adjuvant, Freund's complete adjuvant, a propolis adjuvant, a oil-in-water adjuvant, a cytokine, CpGDNA, a genetically engineered toxoid, an immune-stimulating complex, and a liposome.

According to the varicella-zoster vaccine disclosed herein, the oil-in-water adjuvant is a squalene-containing squalene-based adjuvant. In mouse experiments, when administered at 0.5 μg/dose, the immunogenic complex (e.g., VZV gE-NPM) can achieve a good immune effect when used in combination with 25 μL/dose of the squalene-containing squalene-based adjuvant.

According to the varicella-zoster vaccine disclosed herein, the vaccine comprises, per unit dose for human use, 5-50 μg, preferably 5 μg, 25 μg, or 50 μg of the immunogenic complex and 0.105-10.5 mg of squalene.

The squalene-based adjuvant disclosed herein comprises: (w/w) 0.5%-5% squalene, 0.05%-1% Span 85, 0.05%-1% Tween 80, and 10 mM citrate buffer.

The squalene-based adjuvant disclosed herein comprises: (w/w) 1.5%-5% squalene, 0.05%-1% Span 85, 0.05%-1% Tween 80, and 10 mM citrate buffer.

The squalene-based adjuvant disclosed herein preferably comprises: (w/w) 2%-4.5% squalene, 0.2%-0.5% Span 85, 0.2%-0.5% Tween 80, and 10 mM citrate buffer. The amount of the squalene used is more preferably 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, or 4.4% (w/w), the Span 85 is more preferably 0.3%-0.4% (w/w), and the Tween 80 is more preferably 0.3%-0.4% (w/w).

The squalene-based oil-in-water adjuvant used in specific embodiments disclosed herein may comprise the following components: (w/w) 3.9% squalene, 0.47% Span 85, 0.47% Tween 80, and 10 mM citrate buffer.

The squalene-based oil-in-water adjuvant used in specific embodiments disclosed herein may comprise the following components: (w/w) 4.3% squalene, 0.5% Span 85, 0.5% Tween 80, and 10 mM citrate buffer.

The squalene-based oil-in-water adjuvant used in specific embodiments disclosed herein may comprise the following components: (w/w) 4.03% squalene, 0.5% Span 85, 0.5% Tween 80, 0.016% citric acid, and 0.264% sodium citrate.

The squalene-based oil-in-water adjuvant used in specific embodiments disclosed herein may comprise the following components: (w/w) 3.0225% squalene, 0.375% Span 85, 0.375% Tween 80, 0.012% citric acid, and 0.198% sodium citrate.

The squalene-based oil-in-water adjuvant used in specific embodiments disclosed herein may comprise the following components: (w/w) 2.015% squalene, 0.25% Span 85, 0.25% Tween 80, 0.08% citric acid, and 0.132% sodium citrate.

The squalene-based oil-in-water adjuvant used in specific embodiments disclosed herein may comprise the following components: (w/w) 0.403% squalene, 0.05% Span 85, 0.05% Tween 80, 0.0016% citric acid, and 0.0264% sodium citrate.

Further, the squalene-based adjuvant (adjuvant 1) used in specific embodiments disclosed herein preferably comprises the following components: 10.50 mg of squalene (4.2%), 1.25 mg of Span 85 (0.5%), 1.25 mg of Tween 80 (0.5%), 0.04 mg of citric acid (0.264%), and 0.66 mg of sodium citrate (0.016%) (w/w). The adjuvant can be used in combination with the injection formula of VZV gE-NPM. Optionally, the amount of the adjuvant 1 per unit dose vaccine for mouse experiments may be 25 μL/dose, and the amount of the adjuvant 1 per unit dose vaccine for human use may be 250 μL/dose (0.25 mL/dose).

As described above, the amount of the immunogenic complex VZV gE-NPM and the adjuvant used in humans and mice is different, and the correspondence is as follows: when used in humans, the amount of VZV gE-NPM and that of the adjuvant are both 10 times the amount used in mice, e.g., 5 μg/dose of VZV gE-NPM for mice and 50 μg/dose for humans, 50 μL/dose of adjuvant for mice and 500 μL/dose (0.5 mL/dose) of adjuvant for humans, 25 μL/dose of adjuvant for mice and 250 μg/dose (0.25 mL/dose) of adjuvant for humans, and so on.

The control vaccine in the present disclosure involves an adjuvant AS01B, and the AS01B comprises the following components: 50 μg of quillaja saponin QS-21, 50 μg of 3-O-deacyl-4′-monophosphoryl lipid A (MPL), 1 mg of dioleoyl phosphatidylcholine (DOPC), 0.25 mg of cholesterol, 4.385 mg of sodium chloride, 0.15 mg of anhydrous disodium hydrogen phosphate, and 0.54 mg of potassium dihydrogen phosphate, per 0.5 mL of the AS01B adjuvant. The AS01B adjuvant used in the present disclosure is an adjuvant product sold together with the VZV gE protein in the launched vaccine Shingrix® of GSK.

The present disclosure also provides a kit, which comprises the varicella-zoster vaccine disclosed herein and an instrument and a container required for vaccination of the vaccine.

The present disclosure provides a varicella-zoster vaccine, which comprises a VZV gE-NPM immune composition (i.e., an immune combination comprising VZV gE-NPM, which can be manufactured into a lyophilized formulation or an injection formulation) and an adjuvant (which is a liquid). The VZV gE-NPM immune composition and the adjuvant are packaged in separate vials. The components of the adjuvant comprise 10.50 mg of squalene, 1.25 mg of Span 85, 1.25 mg of Tween 80, 0.04 mg of sodium citrate, and 0.66 mg of citric acid. When the adjuvant is used in combination with the VZV gE-NPM lyophilized formulation, compared with the case of being used in combination with the VZV gE-NPM injection formulation, the concentration of each component in the adjuvant vial for the former case is half (1:1 dilution) of that for the latter case. The content of the VZV gE-NPM immune complex is 50 μg/dose or 25 μg/dose (two strengths).

In the case that the VZV gE-NPM immune composition is in the form of a lyophilized formulation, all liquid needs to be drawn up from the adjuvant vial and transferred into a vial containing the VZV gE-NPM lyophilized formulation before clinical inoculation, and the mixture is thoroughly mixed before use. The human dose after reconstitution is 0.5 mL per administration, which contains 50 μg or 25 μg of the VZV gE-NPM immunogenic complex.

In the case that the VZV gE-NPM immune composition is in the form of an injection formulation, the human dose of the VZV gE-NPM injection is 0.25 mL per administration, and the human dose of the adjuvant is 0.25 mL per administration. All liquid needs to be drawn up from the adjuvant vial and transferred into a vial containing the VZV gE-NPM injection before clinical inoculation, and the mixture is thoroughly mixed before use. The human dose after reconstitution is 0.5 mL per administration, which contains 50 μg or 25 μg of the VZV gE-NPM immunogenic complex.

The present disclosure provides use of the varicella-zoster nanoparticle immunogenic complex, the immune composition, or the vaccine in the manufacture of a medicament for preventing or treating herpes zoster.

All reagents used in the present disclosure are commercially available.

Compared with the prior art, the present disclosure has the following beneficial effects:
(1) The immunogenic complex disclosed herein fills the gap where nanoparticle-based varicella-zoster vaccines are almost unavailable around the world. After trying with different recombinant particle proteins, it has been found that the varicella-zoster nanoparticle immunogenic complexes VZV gE-NPM, VZV gE-I53-50, VZV gE-AP205, and VZV gE-Ferritin obtained using the different nanoparticle proteins NPM, I53-50, AP205, and Ferritin used in the present disclosure can all achieve relatively ideal technical effects, including a uniform particle size, a homogeneous and aggregation-free distribution, stable product performance, and compliant endotoxin levels, all of which demonstrate that they are suitable for non-clinical development and antibody immunogenicity assay and thereby are suitable for use as varicella-zoster vaccines.
(2) Compared with the currently commercially available recombinant protein varicella-zoster vaccine Shingrix®, the varicella-zoster vaccine disclosed herein further improves the level of antibodies produced in the body and the immune effect of T cells. After the varicella-zoster nanoparticle immunogenic complex provided herein was successfully prepared, different non-clinical cell and antibody immunogenicity experimental tests were performed for comparison with Shingrix®. Both the immunogenicity of T cells and the immunogenicity of antibodies induced by the varicella-zoster nanoparticle immunogenic complex provided herein are higher than those induced by Shingrix®, indicating that the varicella-zoster nanoparticle immunogenic complex provided herein can induce cellular immunity and humoral immune responses superior to those induced by the commercialized vaccine Shingrix®. When used in combination with a specific squalene-based adjuvant, the varicella-zoster nanoparticle immunogenic complex provided herein allows the immune system to be involved in the induction process earlier than the commercially available varicella-zoster recombinant protein vaccine Shingrix® of GSK does, resulting in a better immune protection effect.

More importantly, the varicella-zoster nanoparticle immunogenic complex provided herein can still achieve a similar effect to Shingrix® even when the amount of the immunogenic complex used is significantly reduced. For example, after primary immunization with an attenuated varicella vaccine (mimicking infection), mice were then sequentially subjected to two booster immunizations. VZV gE-NPM can induce an excellent humoral immune response, and can induce IFN-γ and IL-2 levels significantly higher than those in the control group at the same antigen dose. A 1/10 dose (i.e., 0.5 μg) of VZV gE-NPM is sufficient to achieve efficacy comparable to that of the full dose (i.e., 5 μg) of Shingrix.
(3) In the present disclosure, a specific linker peptide (EAAAK)₃ is selected. This linker peptide not only promotes the expression level of the fusion protein formed by VZV gE and the binding peptide 1, but also promotes the induction of the immunogenic effect by the immune composition. According to the results of Western Blot, SDS-PAGE, immunogenicity assay, and the like, it has been found that using (EAAAK)₃ to link VZV gE to the binding peptide 1 can achieve the most ideal effect.
(4) In the present disclosure, a specific signal peptide MEFGLSWVFLVAIIKGVQC is selected for transfection and expression, which can result in relatively the highest expression level.
(5) In the present disclosure, the squalene-based adjuvants with different squalene contents were each used in combination with VZV gE-NPM. It has been observed that the vaccine provided herein can exert an ideal immunogenic effect at a very low squalene content, such that the amount of expensive squalene used can be greatly reduced, thereby reducing the cost.
(6) In the present disclosure, specific types and proportional amounts of stabilizers, excipients, surfactants, buffering agents, and pH regulators are used to formulate the immunogenic complex into a lyophilized formulation, which can achieve optimal stability.
(7) The preparation method for the nanoparticle-type varicella-zoster vaccine provided herein features low preparation cost and is suitable for large-scale production.

In the present disclosure, the particle protein component is prepared by E. coli fermentation and chromatographic purification, while the VZV gE antigen is prepared by CHO cell reactor culture and chromatographic purification. Both processes are suitable for industrial mass production and have the advantages of high expression levels, stable process and yield, simple operation, and the like. The recombinant particle protein component produced in a single batch can be combined with the VZV gE antigen produced in multiple batches, thereby improving the production efficiency. Compared with the common recombinant protein vaccine, the nanoparticle vaccine disclosed herein provides higher immunoprotection at the same or lower dose, and thus can save the cost of large-scale production.

The preparation method for the recombinant particle protein component provided herein does not require special equipment, is amenable to scale-up, and is suitable for industrial production. The preparation method features a short production time and a simple and stable process and thus can reduce the cost of large-scale industrial production. The protein product prepared by the method for the recombinant particle protein component provided herein effectively reduces the side effects caused by the residual impurities, host proteins, exogenous DNA, antibiotics, bacterial endotoxins, etc., in the particles, improving the safety.

### BRIEF DESCRIPTION OF THE DRAWINGS

FIG. 1 shows the effect of different linker peptides 1 (linker 1) on the sequence expression and immunogenicity of VZV gE-binding peptide 1 protein, where:
panel a shows the supernatant SDS-PAGE results of the protein sequences of VZV gE-binding peptide 1 (VZV gE-4T) with linker peptides 1 of different structures, namely (G₄S)₃, (EAAAK)₃, and no linker, after 8 days of culture expression in CHO cells; the arrow indicates the position of the target protein;
panels b and c show the target protein content in the supernatant of the harvested liquid after transient transfection and stable transfection of CHO cells for expression, respectively (as measured by ELISA);
panels d, e, and f show the immune test effects of prepared VZV gE-NPM formed by using VZV gE-binding peptide 1 containing different linker peptides 1.

FIG. 2 shows the results of relevant assay of VZV gE-NPM formed by binding of VZV gE to nanoparticle protein NPM, where:
panel a shows SDS-PAGE results of VZV gE-NPM;
panel b shows the Western Blot results (primary antibody is anti-VZV gE antibody) of VZV gE-NPM;
panel c shows the SEC identification results of the sample collected in the VZV gE-NPM purification process.

FIG. 3 shows the results of another relevant assay of VZV gE-NPM formed by binding of VZV gE to nanoparticle protein NPM, where:
panel a shows the SDS-PAGE results of the sample collected in the VZV gE-NPM purification process;
panel b shows the DLS measurement results of the NPM naked particles;
panel c shows the DLS measurement results of VZV gE-NPM.

FIG. 4 shows the results of relevant assay of VZV gE-I53-50 formed by binding of VZV gE to nanoparticle protein I53-50, where:
panel a shows the SEC identification results of VZV gE-I53-50A purified by an SEC column;
panel b shows the SDS-PAGE results of purified VZV gE-I53-50A;
panel c shows the DLS measurement results of the I53-50 naked particles.

FIG. 5 shows the results of another relevant assay of VZV gE-I53-50 formed by binding of VZV gE to nanoparticle protein I53-50, where:
panel a shows the SEC identification results of VZV gE-I53-50 formed after binding;
panel b shows the SDS-PAGE results of purified I53-50A, I53-50B, and VZV gE-I53-50A;
panel c shows the DLS measurement results of VZV gE-I53-50.

FIG. 6 shows the results of relevant assay of VZV gE-Ferritin formed by binding of VZV gE to nanoparticle protein Ferritin, where:
panel a shows the SDS-PAGE identification results of purified Ferritin-4C;
panel b shows the SDS-PAGE identification results of purified VZV gE-Ferritin;
panel c shows the DLS measurement results of VZV gE-Ferritin.

FIG. 7 shows the identification results and particle size measurement results of purified VZV gE-AP205 binding product, where:
panel a shows the SDS-PAGE identification results of purified AP205-4C formed after binding;
panel b shows the DLS measurement results of VZV gE-AP205.

FIG. 8 shows the electron microscope examination results of VZV gE-NPM.

FIG. 9 shows the electron microscope examination results of VZV gE-I53-50.

FIG. 10 shows the electron microscope examination results of VZV gE-Ferritin.

FIG. 11 shows the electron microscope examination results of VZV gE-AP205.

FIG. 12 shows the cellular and humoral immune responses of four particle vaccines VZV gE-NPM, VZV gE-I53-50, VZV gE-Ferritin, and VZV gE-AP205 in a mouse model, where:
panel a shows the IgG antibody titer produced by the four nanoparticle vaccines in the mouse model on day 13 after immunization;
panel b shows the IgG antibody titer produced by the four nanoparticle vaccines in the mouse model on day 28 after immunization;
panel c shows the assay results of spleen cytokine IL-2 produced by the four nanoparticle vaccines in the mouse model on day 28 after immunization;
panel d shows the assay results of spleen cytokine IFN-γ produced by the four nanoparticle vaccines in the mouse model on day 28 after immunization.

FIG. 13 shows cellular and humoral immune responses induced by the VZV gE-NPM particle vaccine in a mouse model subjected to primary immunization with a varicella attenuated vaccine, where: panel a shows the IgG antibody titer produced by VZV gE-NPM in the mouse model on day 58 after immunization;
panel b shows the assay results of spleen cytokine IFN-γ produced by VZV gE-NPM in the mouse model on day 58 after immunization;
panel c shows the assay results of spleen cytokine IL-2 produced by VZV gE-NPM in the mouse model on day 58 after immunization.

FIG. 14 shows the results of immune responses induced by VZV gE-NPM particle vaccine in combination with adjuvants with different squalene contents.

### DETAILED DESCRIPTION

The principles and features disclosed herein are described with reference to the following examples, and the examples provided are only intended to explain the present disclosure and are not intended to limit the scope disclosed herein. Before the detailed description disclosed herein is further provided, it should be understood that the protection scope disclosed herein is not limited to the specific embodiments described below; it should also be understood that the terminology used in the examples herein is intended to describe specific embodiments and is not intended to limit the protection scope disclosed herein. Test procedures without specified conditions in the following examples are generally conducted according to conventional conditions or according to conditions recommended by manufacturers. When numerical ranges are given in the examples, it should be understood that, unless otherwise specified in the present disclosure, both endpoints of each of the numerical ranges and any numerical value between the two endpoints can be selected. Unless otherwise defined, all technical and scientific terms used herein have the same meaning as commonly understood by those skilled in the art. In addition to the specific methods, equipment, and materials used in the examples, any methods, equipment, and materials similar or equivalent to those described in the examples herein can also be used to implement the present disclosure, based on the understanding of the prior art by those skilled in the art and the disclosure disclosed herein. The experimental materials used in the following examples were purchased from conventional reagent companies, unless otherwise specified.

### Example 1. Determination of Linker Peptide 1 and Signal Peptide

### 1. Materials-Linker Peptide 1 and Signal Peptide

(1) Linker peptide 1:
Design 1-without linker peptide 1 (no linker 1);
design 2-
(SEQ ID NO: 3)
GGGGSGGGGSGGGGS;
design 3-
(SEQ ID NO: 4)
EAAAKEAAAKEAAAK
(2) Signal peptide:
(SEQ ID NO: 10)
MGWSLILLFLVAVATRVLS,
(SEQ ID NO: 11)
MEWSWVFLFFLSVTTGVHS,
(SEQ ID NO: 12)
MEFGLSWVFLVAIIKGVQC

### 2. Experimental Method

### (1) Confirmation Experiment in the Case of Using No Linker Peptide 1 or Using Linker Peptides 1 of Different Structures:

The sequence of the first 544 amino acids of the VZV gE protein falls outside the transmembrane domain and is antigenic. Therefore, the VZV gE vaccine was designed based on the sequence of amino acids at positions 1-544. The original signal peptide consisting of the amino acids at positions 1-30 was replaced with the specific signal peptide set forth in SEQ ID NO: 12 selected and determined in the present disclosure, and the specific signal peptide was linked to the extracellular region of the varicella-zoster virus (VZV) gE protein (VZV gE31-544) set forth in SEQ ID NO: 14, the extracellular region was then linked to the binding peptide 1 set forth in SEQ ID NO: 1 (i.e., “4T”) via the linker peptide 1 (linker 1) set forth in SEQ ID NO: 3 or SEQ ID NO: 4 to form a fusion protein, and meanwhile, a histidine 6His purification tag was added to the C-terminus of the fusion protein. The encoding gene encoding the above fusion protein was inserted into a eukaryotic cell expression vector pcDNA3.4 for expression in CHO cells, thus obtaining the fusion protein VZV gE-binding peptide 1, i.e., VZV gE-4T, as an antigenic component.

### (2) Confirmation Experiment in the Case of Using Different Signal Peptides:

The linker peptide 1 was set forth in SEQ ID NO: 3, the signal peptide was set forth in SEQ ID NO: 10, SEQ ID NO: 11, or SEQ ID NO: 12, the signal peptide was linked to the N-terminus of VZV gE31-544, and VZV gE-4T and VZV gE-I53-50A were expressed. Details are as follows: Preparation of VZV gE-4T: VZV gE31-544 was set forth in SEQ ID NO: 14, the N-terminus of VZV gE31-544 was linked to the signal peptide set forth in SEQ ID NO: 10, SEQ ID NO: 11, or SEQ ID NO: 12, and the C-terminus of VZV gE31-544 was linked to the linker peptide 1 set forth in SEQ ID NO: 3, such that VZV gE was linked to the binding peptide 1 (the binding peptide 1 was named “4T”) at the C-terminus, and meanwhile, a 6His purification tag was added to the C-terminus of the fusion protein. The encoding gene encoding the above fusion protein was inserted into a eukaryotic cell expression vector pcDNA3.4 for expression in CHO cells, thus obtaining the fusion protein VZV gE-4T as an antigenic component.

Preparation of VZV gE-I53-50A: VZV gE31-544 was set forth in SEQ ID NO: 14, the N-terminus of VZV gE31-544 was linked to the signal peptide set forth in SEQ ID NO: 10, SEQ ID NO: 11, or SEQ ID NO: 12, then VZV gE31-544 was fused to the I53-50A set forth in SEQ ID NO: 19 via the linker peptide 1 set forth in SEQ ID NO: 3, and the encoding gene encoding the above fusion protein was inserted into a eukaryotic cell expression vector pcDNA3.4 for expression in CHO-S cells, thus obtaining the fusion protein VZV gE-I53-50A as an antigenic component.

### 3. Experimental Results

### (1) Experimental Results in the Case of Using Different Linker Peptides 1:

When three designs (no linker, (G₄S)₃, and (EAAAK)₃) were selected to transiently transfect CHO cells to express VZV gE-4T, the target antigen was expressed, as shown in panel a in FIG. 1 and panel b in FIG. 1, where panel a in FIG. 1 shows the SDS-PAGE identification result of the CHO transient transfection expression supernatant, and panel b in FIG. 1 shows the identification result of the above expression supernatant by ELISA. The results showed that all three designs achieved transient transfection and expression in CHO cells, where the expression level obtained with (EAAAK)₃ was the highest, that with no linker was the second highest, and that with (G₄S)₃ linker was the lowest.

Further, as shown in panel b in FIG. 1 and panel c in FIG. 1, under the expression conditions for transient transfection and stable transfection in CHO cells, the expression supernatant was subjected to identification of the target protein content by ELISA, and the results were consistent with the SDS-PAGE trend shown in panel a in FIG. 1. The expression level of the target product obtained when (EAAAK)₃ was selected as the linker was the best, and was similar to that obtained when no linker was present. When (G₄S)₃ was used as the linker, the expression level of the target product was not as high as that in the case where no linker was present or (EAAAK) 3 was used as the linker.

After VZV gE-NPM was prepared by using linker1 with different structures to prepare VZV gE-4T and allowing VZV gE-4T to bind to NPM-4C, immune tests were performed in animals and corresponding effects were obtained. As shown in panel d in FIG. 1, panel e in FIG. 1, and panel f in FIG. 1 (see Example 10 for details), the use of (EAAAK)₃ obtained the best immunological effects, (EAAAK)₃ was superior to (G₄S)₃, and the designs using (EAAAK)₃ and (G₄S)₃ were both superior to the design with no linker.

In conclusion, in the present disclosure, (EAAAK)₃ and (G₄S)₃ are used to link VZV gE and the binding peptide 1 (i.e., 4T), and (EAAAK)₃ (SEQ ID NO: 4) is most preferably used to prepare a fusion protein VZV gE-binding peptide 1 (i.e., VZV gE-4T).

### (2) Experimental Results in the Case of Using Different Signal Peptides:

The different signal peptides above were used in the experiment. According to the transient transfection results, the signal peptide set forth in SEQ ID NO: 12 resulted in the highest expression level. Therefore, in the present disclosure, the signal peptide set forth in SEQ ID NO: 12 was used for stable transfection.

TABLE 1
Assay of expression levels of target proteins with different signal peptides

| | | Transient | Expression | | |
|---|---|---|---|---|---|
| Target | Signal | transfection | level | | |
| protein | peptide | volume | ELISA) | | Results |
| VZV gE-4T | SEQ ID NO: 12 | 30 mL | 67 | mg/L | According to the transient transfection results, |
| | | | | | the signal peptide set forth in SEQ ID NO: 12 |
| | SEQ ID NO: 11 | 30 mL | 20 | mg/L | resulted in the highest expression level, so this |
| | SEQ ID NO: 10 | 30 mL | 44 | mg/L | signal peptide was used for stable transfection. |
| VZV gE-I53-50A | SEQ ID NO: 12 | 30 mL | 200 | mg/L | According to the transient transfection results, |
| | SEQ ID NO: 11 | 30 mL | 40 | mg/L | the signal peptide set forth in SEQ ID NO: 12 |
| | | | | | resulted in the highest expression level, so this |
| | SEQ ID NO: 10 | 30 mL | 65 | mg/L | signal peptide was used for stable transfection. |

TABLE 2
Amino acid sequences of binding peptides, linker peptides, and
signal peptides in examples of the present application

| Polypeptide name | Amino acid sequence |
|---|---|
| Binding peptide 1 | AHIVMVDAYKPTK (SEQ ID NO: 1) |
| (4T) | |
| Binding peptide 2 | MDSATHIKFSKRDEDGKELAGATMELRDSSGKTISTWISDGQVKDFYLYPGKYTFVE |
| (4C) | TAAPDGYEVATAITFTVNEQGQVTVNGKATKGDAHI (SEQ ID NO: 2) |
| Linker peptide 1 | GGGGSGGGGSGGGGS (SEQ ID NO: 3) |
| | EAAAKEAAAKEAAAK (SEQ ID NO: 4) |
| | GGSGGSGSEKAAKAEEAAR (SEQ ID NO: 5) |
| Linker peptide 2 | GGSGGSGGS (SEQ ID NO: 6) |
| | GGSGGSGGSGGS (SEQ ID NO: 7) |
| | SGGSGG (SEQ ID NO: 8) |
| | GSGGSGGSG (SEQ ID NO: 9) |
| Signal peptide | MGWSLILLFLVAVATRVLS (SEQ ID NO: 10) |
| | MEWSWVFLFFLSVTTGVHS (SEQ ID NO: 11) |
| | MEFGLSWVFLVAIIKGVQC (SEQ ID NO: 12) |
| VZV gE | MGTVNKPVVGVLMGFGIITGTLRITNPVRASVLRYDDFHIDEDKLDTNSVYEPYYHS |
| (positions 1-544) | DHAESSWVNRGESSRKAYDHNSPYIWPRNDYDGFLENAHEHHGVYNQGRGIDSGE |
| | RLMQPTQMSAQEDLGDDTGIHVIPTLNGDDRHKIVNVDQRQYGDVFKGDLNPKPQ |
| | GQRLIEVSVEENHPFTLRAPIQRIYGVRYTETWSFLPSLTCTGDAAPAIQHICLKHTTC |
| | FQDVVVDVDCAENTKEDQLAEISYRFQGKKEADQPWIVVNTSTLFDELELDPPEIEP |
| | GVLKVLRTEKQYLGVYIWNMRGSDGTSTYATFLVTWKGDEKTRNPTPAVTPQPRGA |
| | EFHMWNYHSHVFSVGDTFSLAMHLQYKIHEAPFDLLLEWLYVPIDPTCQPMRLYST |
| | CLYHPNAPQCLSHMNSGCTFTSPHLAQRVASTVYQNCEHADNYTAYCLGISHMEPSF |
| | GLILHDGGTTLKFVDTPESLSGLYVFVVYFNGHVEAVAYTVVSTVDHFVNAIEERGF |
| | PPTAGQPPATTKPKEITPVNPGTSPLIRYAAWTGG |
| | (SEQ ID NO: 13) |
| VZV gE | SVLRYDDFHIDEDKLDTNSVYEPYYHSDHAESSWVNRGESSRKAYDHNSPYIWPRN |
| (positions 31-544) | DYDGFLENAHEHHGVYNQGRGIDSGERLMQPTQMSAQEDLGDDTGIHVIPTLNGD |
| | DRHKIVNVDQRQYGDVFKGDLNPKPQGQRLIEVSVEENHPFTLRAPIQRIYGVRYTE |
| | TWSFLPSLTCTGDAAPAIQHICLKHTTCFQDVVVDVDCAENTKEDQLAEISYRFQGK |
| | KEADQPWIVVNTSTLFDELELDPPEIEPGVLKVLRTEKQYLGVYIWNMRGSDGTSTY |
| | ATFLVTWKGDEKTRNPTPAVTPQPRGAEFHMWNYHSHVFSVGDTFSLAMHLQYKI |
| | HEAPFDLLLEWLYVPIDPTCQPMRLYSTCLYHPNAPQCLSHMNSGCTFTSPHLAQRV |
| | ASTVYQNCEHADNYTAYCLGISHMEPSFGLILHDGGTTLKFVDTPESLSGLYVFVVY |
| | FNGHVEAVAYTVVSTVDHFVNAIEERGFPPTAGQPPATTKPKEITPVNPGTSPLIRYAA |
| | WTGG |
| | (SEQ ID NO: 14) |

### Example 2. Expression of Encoding Gene of VZV gE-Binding Peptide 1 Fusion Protein

The original signal peptide consisting of the amino acids at positions 1-30 was replaced with the specific signal peptide set forth in SEQ ID NO: 12 selected and determined in the present disclosure, the specific signal peptide was linked to the extracellular region of the varicella-zoster virus (VZV) gE protein (VZV gE31-544) set forth in SEQ ID NO: 14 (the signal peptide was linked to the N-terminus of VZV gE31-544), the extracellular region was then linked to the binding peptide 1 (i.e., “4T”) set forth in SEQ ID NO: 1 via the linker peptide 1 (linker 1) (G₄S)₃ (set forth in SEQ ID NO: 3) or (EAAAK)₃ (set forth in SEQ ID NO: 4), and meanwhile, a histidine 6His purification tag was added to the C-terminus. The encoding gene encoding the above fusion protein was inserted into a eukaryotic cell expression vector pcDNA3.4 for expression in CHO cells, thus obtaining the fusion protein VZV gE-binding peptide 1, i.e., “VZV gE-4T”, as an antigenic component. The selection and determination procedures for the signal peptide and linker peptide 1 are shown in Example 1. The signal peptide linked to the N-terminus of VZV gE31-544 generated during intracellular expression was cleaved off in the host cells, and VZV gE-4T secreted from the host cells contained no signal peptide.

The VZV gE-4T (without signal peptide) formed using the linker peptide 1 set forth in SEQ ID NO: 4 was set forth in SEQ ID NO: 15, and the VZV gE-4T (without signal peptide) formed using the linker peptide 1 set forth in SEQ ID NO: 3 was set forth in SEQ ID NO: 16, as shown in Table 3.

TABLE 3
Sequences of VZV gE-binding peptide 1 fusion
proteins in examples of the present
application

Amino acid sequence
VZV gE-4T:
SVLRYDDFHIDEDKLDTNSVYEPYYHSDHAESSWVNRGESSRKAY
DHNSPYIWPRNDYDGFLENAHEHHGVYNQGRGIDSGERLMQPTQM
SAQEDLGDDTGIHVIPTLNGDDRHKIVNVDQRQYGDVFKGDLNPK
PQGQRLIEVSVEENHPFTLRAPIQRIYGVRYTETWSFLPSLTCTG
DAAPAIQHICLKHTTCFQDVVVDVDCAENTKEDQLAEISYRFQGK
KEADQPWIVVNTSTLFDELELDPPEIEPGVLKVLRTEKQYLGVYI
WNMRGSDGTSTYATFLVTWKGDEKTRNPTPAVTPQPRGAEFHMWN
YHSHVFSVGDTFSLAMHLQYKIHEAPFDLLLEWLYVPIDPTCQPM
RLYSTCLYHPNAPQCLSHMNSGCTFTSPHLAQRVASTVYQNCEHA
DNYTAYCLGISHMEPSFGLILHDGGTTLKFVDTPESLSGLYVFVV
YFNGHVEAVAYTVVSTVDHFVNAIEERGFPPTAGQPPATTKPKEI
TPVNPGTSPLIRYAAWTGGEAAAKEAAAKEAAAKAHIVMVDAYKP
TKHHHHHH
(SEQ ID NO: 15)
VZV gE-4T:
SVLRYDDFHIDEDKLDTNSVYEPYYHSDHAESSWVNRGESSRKAY
DHNSPYIWPRNDYDGFLENAHEHHGVYNQGRGIDSGERLMQPTQM
SAQEDLGDDTGIHVIPTLNGDDRHKIVNVDQRQYGDVFKGDLNPK
PQGQRLIEVSVEENHPFTLRAPIQRIYGVRYTETWSFLPSLTCTG
DAAPAIQHICLKHTTCFQDVVVDVDCAENTKEDQLAEISYRFQGK
KEADQPWIVVNTSTLFDELELDPPEIEPGVLKVLRTEKQYLGVYI
WNMRGSDGTSTYATFLVTWKGDEKTRNPTPAVTPQPRGAEFHMWN
YHSHVFSVGDTFSLAMHLQYKIHEAPFDLLLEWLYVPIDPTCQPM
RLYSTCLYHPNAPQCLSHMNSGCTFTSPHLAQRVASTVYQNCEHA
DNYTAYCLGISHMEPSFGLILHDGGTTLKFVDTPESLSGLYVFVV
YFNGHVEAVAYTVVSTVDHFVNAIEERGFPPTAGQPPATTKPKEI
TPVNPGTSPLIRYAAWTGGGGGSGGGSGGGSAHIVMVDAYKPTKH
HHHHH
(SEQ ID NO: 16)

### Example 3: Purification of VZV gE-Binding Peptide 1 Fusion Protein

The fusion protein VZV gE-4T (set forth in SEQ ID NO: 15 or SEQ ID NO: 16) antigenic component obtained by the expression in Example 2 was purified by nickel column affinity chromatography and size exclusion chromatography to obtain a high-purity protein. The specific procedures are as follows:

### 1. Experimental Materials

capsule filter (Bricap C01: 180 cm²) purchased from Cobetter, Pellicon2 ultrafiltration membrane cassette purchased from Millipore, nickel ion affinity packing Ni Bestarose FF purchased from Bestchrom, SEC column (HiLoad 16/600 Superdex 200 μg) purchased from Cytiva, and ultrafiltration fixture and peristaltic pump Masterflex L/S

Consumables for ultrafiltration: Millipore 10 kDa Pellicon2 regenerated cellulose micromembrane (0.1 m² surface area)

### 2. Experimental Method

### (1) Sample Treatment:

The CHO cell culture supernatant containing the above VZV gE-binding peptide 1 fusion protein was centrifuged at high speed in a centrifuge at 12000 g for 60 min, and about 2.35 L of supernatant was collected. Then, a Cobetter 0.45+0.2 μm filter (model: Bricap COT: 180 cm²) was used for clarification filtration.

### (2) Tangential Flow Filtration

This step was mainly intended to concentrate the above clarified feed liquid and allow it to be buffer-exchanged into the buffer for affinity chromatography to reduce the influence of EDTA in the culture medium on the nickel affinity packing, and the procedure was performed at room temperature.

Ultrafiltration devices used and related methods are as follows:

Ultrafiltration process: The membrane cassette was washed with water, followed by a 30-minute cleaning and sanitization cycle with 0.1 M sodium hydroxide solution at a peristaltic pump flow rate of 400 mL/min. After flushing with 6 L of water, the pH was measured at approximately 10. The ultrafiltration membrane cassette was then equilibrated by rinsing with 1 L of TBS buffer (20 mM Tris-HCl, 150 mM NaCl, pH 7.4). 1 L of TBS buffer was then used for equilibration of the membrane cassette, and the pH of the permeate reached 7.38.

Concentration of VZV gE-4T culture supernatant: Approximately 2.3 L of the above clarified feed liquid was concentrated at a peristaltic pump flow rate of 500 mL/min, a sample agitating speed of approximately 150 r/min, a feed port pressure of 13 psi, and a retentate port pressure of 5 psi (with a permeate flow rate of approximately 52 mL/min). Using these parameters, the feed liquid was concentrated to 0.46 L. Buffer exchange was then performed. A 6-fold diafiltration was performed using 2.8 L of TBS buffer at a feed port pressure of 12 psi and a retentate port pressure of 5 psi.

Harvest of concentrated sample: The permeate outlet was closed. With the peristaltic pump set at a flow rate of 200 mL/min, a total of approximately 0.5 L of VZV gE-4T ultrafiltration component was harvested. The ultrafiltration membrane cassette was subsequently subjected to sanitization with water and a 60-min sanitization cycle with 0.1 M sodium hydroxide, and it was finally stored in 0.1 M sodium hydroxide.

### (3) Nickel Ion Affinity Chromatography

Affinity purification was accomplished using a nickel ion affinity packing chromatography column. The volume of the chromatography column was 10 mL, the flow rate for chromatography was 5 mL/min, and 70 mL of ultrafiltration sample was loaded.

Chromatography procedure: The chromatography column was subjected to CIP and rinsing, and then the column was equilibrated with TBS (20 mM Tris-HCl, 150 mM NaCl, pH 7.4), followed by sample loading. The column was washed with TBS buffer, and then protein impurities were removed by washing with 20 mM imidazole in TBS buffer. Finally, the VZV gE-binding peptide 1 (i.e., VZV gE-4T) component was eluted with 500 mM imidazole in TBS buffer.

### (4) Size Exclusion Purification

Purification was performed using a HiLoad 16/600 Superdex 200 μg column. The column volume of the SEC column was 120 mL, and the sample load amount of the VZV gE-4T affinity-purified sample was controlled at approximately 4% of the column volume.

Chromatography procedure: The chromatography column was subjected to CIP and rinsing, and then the column was equilibrated with TBS (20 mM Tris-HCl, 150 mM NaCl, pH 7.4), followed by sample loading. The column was eluted with TBS buffer, and the VZV gE-4T component was collected.

### Example 4: Expression and Purification of Binding Peptide 2-NPM Fusion Protein

The nanoparticle protein NPM (set forth in SEQ ID NO: 17) was linked, at its N-terminus, to the binding peptide 2 (set forth in SEQ ID NO: 2, sequence shown in Table 1) via the linker peptide 2 (set forth in SEQ ID NO: 7, sequence shown in Table 1) to form a binding peptide 2-NPM fusion protein, i.e., NPM-4C (set forth in SEQ ID NO: 18). The related sequences of NPM and NPM-4C are shown in Table 4.

TABLE 4
Sequences of NPM and NPM-4C fusion protein
in examples of the present application

| Polypeptide | |
|---|---|
| name | Amino acid sequence |
| NPM | MKMEELFKKHKIVAVLRANSVEEAKKKALA |
| | VFLGGVHLIEITFTVPDADTVIKELSFLKE |
| | MGAIIGAGTVTSVEQARKAVESGAEFIVSP |
| | HLDEEISQFAKEKGVFYMPGVMTPTELVKA |
| | MKLGHTILKLFPGEVVGPQFVKAMKGPFPN |
| | VKFVPTGGVNLDNVCEWFKAGVLAVGVGSA |
| | LVKGTPVEVAEKAKAFVEKIRGCTE |
| | (SEQ ID NO: 17) |
| NPM-4C | MDSATHIKFSKRDEDGKELAGATMELRDSS |
| | GKTISTWISDGQVKDFYLYPGKYTFVETAA |
| | PDGYEVATAITFTVNEQGQVTVNGKATKGD |
| | AHIGGSGGSGGSGGSMKMEELFKKHKIVAV |
| | LRANSVEEAKKKALAVFLGGVHLIEITFTV |
| | PDADTVIKELSFLKEMGAIIGAGTVTSVEQ |
| | ARKAVESGAEFIVSPHLDEEISQFAKEKGV |
| | FYMPGVMTPTELVKAMKLGHTILKLFPGEV |
| | VGPQFVKAMKGPFPNVKFVPTGGVNLDNVC |
| | EWFKAGVLAVGVGSALVKGTPVEVAEKAKA |
| | FVEKIRGCTE |
| | (SEQ ID NO: 18) |

The encoding gene of the fusion protein was expressed in E. coli. The bacterial cells were harvested and then disrupted through high-pressure homogenization to release the target protein. The feed liquid was clarified, with the main purpose of removing bacterial cell debris and protein impurities. The clarification of the feed liquid was mainly achieved by heating treatment. The heating treatment was performed using a two-step heating method. The E. coli lysate supernatant was subjected to first-step heating and second-step heating (i.e., “two-step heating”). The impurity removal effect and the purity of the recombinant particle protein component in the two-step heating process were measured. 60 g of wet E. coli bacterial cells collected by centrifugation were taken, resuspended in 240 mL of a buffer (20 mM Tris-HCl, 2 mM PMSF, pH 9.0), and disrupted using a high-pressure homogenizer at 1000 bar. After centrifugation, 280 mL of supernatant was collected. 40 mL of the resulting supernatant was then taken and subjected to the two-step heating operation. The lysate supernatant, the supernatant obtained after the first-step heating and centrifugation, and the resuspension of pellet obtained after the second-step heating and centrifugation were subjected to SDS-PAGE analysis. As shown in Table 5, in the first-step heating treatment, the pH was adjusted to 9.0, and the sample was heated in a water bath at 80° C. for 1 h. After returning to room temperature, centrifugation was performed to collect a supernatant (about 35 mL). In the second-step heating treatment, 35 mL of a pH 7.4 buffer containing 100 mM Tris-HCl, 5 mM EDTA, and 4% Triton was added, and then 7 mL of 1 M Tris-HCl pH 7.4 was added. The mixture was thoroughly mixed, heated in a water bath at 60° C. for 10 min, and then immediately centrifuged to collect a pellet. The pellet was then redissolved in a pH 9.0 buffer containing 20 mM Tris-HCl and 5 mM EDTA.

TABLE 5
Two-step heating extraction process for recombinant
particle protein component product

| Procedure | Reaction condition | Operational value | |
|---|---|---|---|
| First pelleting | Heating temperature | 80° | C. |
| and | Heating duration | 60 | min |
| centrifugation | Centrifugation rotation | 12000 | g |
| | speed | | |
| | Centrifugation | 4° | C. |
| | temperature | | |
| | Centrifugation duration | 30 | min |
| Supernatant | Dilution buffer | 100 mM Tris-HCl, 5 mM | |
| dilution | | EDTA, 4% Triton, pH 7.4 | |
| | Volume of dilution | 1:1 | (v/v) |
| | buffer | | |
| | pH buffer | 1M Tris-HCl, pH 7.4 | |
| | Volume of pH buffer | 10% of the total volume | |
| Second | Heating temperature | 60° | C. |
| pelleting and | Heating duration | 10 | min |
| centrifugation | Centrifugation rotation | 6000 | g |
| | speed | | |
| | Centrifugation | 30° | C. |
| | temperature | | |
| | Centrifugation duration | 10 | min |
| Pellet | Resuspension buffer | 20 mM Tris-HCl, | |
| resuspending | | 5 mM EDTA, pH 9.0 | |
| | Volume of | Resuspending to pre- | |
| | resuspension buffer | centrifugation volume | |
| | pH | 9.0 ± 0.1 | |
| | Filtration | 0.22 | μm |

Incorporating urea and sodium chloride at varying concentrations after the two-step heating treatment and prior to chromatographic purification significantly reduced impurities other than the target recombinant particle protein. The optimized pretreatment condition for the recombinant particle protein component before Fractogel DEAF M chromatography was soaking in 8 M urea and 50-200 mM sodium chloride.

The above recombinant particle protein component sample solution was refined by ion exchange chromatography and hydrophobic chromatography. The first-step chromatographic purification was performed using the Fractogel DEAF M chromatography process. The specific steps and parameters are shown in Table 6. The Fractogel DEAF M eluate sample was first diluted with a buffer, and then a 500 (w/v) sucrose stabilizer was added. The specific parameters are shown in Table 7. Then, the sample was further refined using a hydrophobic chromatography process with Octyl Bestarose 4FF (second-step chromatographic purification). The specific procedures and parameters are shown in Table 8.

### Method for First-Step Chromatography: Chromatography Packing-Fractogel DEAF M, Retention Time-12.5 Min

TABLE 6
Method for first-step chromatography

| Chromatography | Chromatography | | |
|---|---|---|---|
| procedures | buffer/condition | Parameter | |
| Equilibration buffer | 20 mM Tris-HCl, 5 mM EDTA, | 6 | CV |
| | 8M Urea, 50 mM NaCl, pH 9.0 | | |
| pH after equilibration | 8.8 ± 0.05 | 8.80 | |
| Rinse buffer 1 | 20 mM Tris-HCl, 5 mM EDTA, | 1.5 | CV |
| | 8M Urea, 50 mM NaCl, pH 9.0 | | |
| Rinse buffer 2 | 20 mM Tris-HCl, 5 mM EDTA, | 5 | CV |
| | 8M Urea, 2% Triton, pH 9.0 | | |
| Rinse buffer 3 | 20 mM Tris-HCl, 8M Urea, pH 9.0 | 5 | CV |
| Rinse buffer 4 | 20 mM Tris-HCl, 4M Urea, pH 9.0 | 5 | CV |
| Elution buffer | 20 mM Tris-HCl, 4M Urea, | 2 | CV |
| | 150 mM, pH 9.0 | | |

| | | |
|---|---|---|
| Collection range | 50 mAU-50 mAU | Optical path |
| | | length of 2 |
| | | mm |

TABLE 7
Method for sample dilution before second-step chromatography

| Procedure | Buffer for dilution | Dilution volume |
|---|---|---|
| Collection of eluate | N/A | N/A |
| from first-step | | |
| chromatography | | |
| Buffer dilution | 20 mM Tris-HCl, | 2× the volume of |
| | 1M NaCl, 50% (w/v) | eluate |
| | sucrose, pH 9.0 | |
| Buffer dilution | 20 mM Tris-HCl, | 1× the volume of |
| | 2M NaCl, pH 9.0 | eluate |

### Method for Second-Step Chromatography: Chromatography Packing-Octyl Bestarose 4FF, Retention Time-12.5 Min

TABLE 8
Method for second-step chromatography

| Chromatography | Chromatography | |
|---|---|---|
| procedures | buffer/condition | Parameter |
| Equilibration buffer | 20 mM Tris-HCl, 1M NaCl, | 2 CV |
| | 25% (w/v) sucrose, pH 9.0 | |
| Rinsing buffer | 20 mM Tris-HCl, 1M NaCl, | 1.5 CV |
| | 25% (w/v) sucrose, pH 9.0 | |
| Elution buffer | 20 mM Tris-HCl, 25% (w/v) | 3 CV |
| | sucrose, pH 9.0 | |
| Collection range | 50 mAU-50 mAU | Optical path |
| | | length of 2 mm |

### Results and Analysis:

Through purity testing, it was found that after further refining using the above chromatography medium combination, the purity of the resulting product reached 99.0% or more.

### Example 5: Binding of VZV gE to NPM and Particle Characterization

### I. Binding of VZV gE to NPM

1. Generation of VZV gE-NPM Binding Product:

The VZV gE-binding peptide 1 (VZV gE-4T) set forth in SEQ ID NO: 15 or SEQ ID NO: 16 and the binding peptide 2-NPM (NPM-4C) set forth in SEQ ID NO: 18 were mixed at ratio of 6:1 based on their BCA protein concentrations, and a 50% (w/v) sucrose stock solution was added until a final sucrose concentration of about 25% (w/v) was achieved. A 1 M Tris-HCl pH 7.4 stock solution equivalent to 1000 of the total reaction volume was added to stabilize the pH. The binding reaction was carried out at 22° C. for 48 h. As an example, the VZV gE-NPM binding system specifically may be: 6 mL of VZV gE-4T (1 mg/mL), 1 mL of NPM-4C (1 mg/mL), 8.75 mL of 50% sucrose, and 1.75 mL of 1 M Tris-HCl 7.4 in a total volume of 17.5 mL.

2. Purification of VZV gE-NPM Binding Product:

The VZV gE-NPM binding product was purified using Cytiva HiLoad 16/600 Superdex 200 μg (column volume 120 mL) or Cytiva Superdex 200 Increase 10/300 GL (column volume 23 mL), and the VZV gE antigen not bound to NPM-4C was isolated and removed. If the SEC column HiLoad 16/600 Superdex 200 μg was used, the sample load amount of the VZV gE-NPM binding sample was controlled at about 3%-6%; if the SEC column Superdex 200 Increase 10/300 GL was used, the sample load amount of the VZV gE-NPM binding sample was controlled at 0.5-1 mL.

Chromatography procedure: The chromatography column was subjected to CIP and rinsing, and then the column was equilibrated with the equilibration buffer (a 12.5% (w/v) sucrose TBS solution containing 20 mM Tris-HCl, 150 mM NaCl, 12.5% (w/v) sucrose, pH7.4), followed by sample loading. The column was eluted with the 12.5% (w/v) sucrose TBS solution, and the VZV gE-NPM component was collected.

### II. Particle Characterization

1. Experimental Material: VZV gE-NPM Particles Prepared in the Above Example

### 2. Assay Method:

### (1) TEM Examination:

Negative staining samples were prepared using a floating method. A 400-mesh grid with a support film was selected and pre-treated to render it hydrophilic. Deionized water and a 2% uranyl formate negative stain solution were prepared. 3 μL of the prepared protein sample (0.12 mg/mL) was directly dripped to one side of the grid where the support film is located. After 1 minute, excess liquid was removed by touching the edge of the grid with clean filter paper. The grid was then briefly air-dried before being sequentially and rapidly rinsed twice on droplets of deionized water. This was followed by a single rinse with 5 μL of negative stain solution. Finally, 5 μL of negative stain solution was dripped to the grid and the grid was allowed to stand for 1 min. Then, the grid was held with tweezers, and the stain solution was removed using filter paper, leaving a thin layer to air-dry naturally before examination. The grid was examined under a 120 kV transmission electron microscope (FERRITINI Tecnai Spirit). The overall staining of the grid was assessed at low magnification. Holes with suitable thickness were selected for observation, and appropriate areas were chosen for imaging and data collection at high magnification.

### (2) SDS-PAGE and Western Blot Methods

For SDS-PAGE, the test sample was prepared using the LDS sample loading buffer (4×) and the reducing agent DTT, heated at 70° C. for 5 min, cooled to room temperature, centrifuged at 10,000 rpm for 20 s, and vortexed for thorough mixing. The final sample load amount was 5 μg. Both the test sample and the non-prestained protein molecular weight standard were loaded onto a 4-12% Bis-Tris gel, and the MES running buffer was applied. Electrophoresis was performed at a voltage of 150 V for approximately 60 min. Following electrophoresis, the gel was removed and placed in a clean container. An appropriate amount of Coomassie brilliant blue staining solution was added to submerge the gel, followed by staining for 2 h on a shaker. After staining, the stain solution was discarded, and the gel was soaked in purified water for destaining on a shaker until the background was clear. The gel was then imaged using a GelDoc Go gel imager. For Western blot, the sample load amount of the test sample was 0.5 μg. The gel electrophoresis method was identical to the SDS-PAGE procedure. Membrane transfer was performed using the Trans-Blot® Turbo instrument and associated reagents. The membrane was incubated with an anti-gE mouse monoclonal antibody and an alkaline phosphatase (AP)-conjugated goat anti-mouse secondary antibody using an iBind instrument. Finally, a chromogenic solution was used for color development and GelDoc Go was used for imaging.

### (3) DLS Method

The purified and prepared test sample was diluted to 0.25 mg/mL. A Zetasizer Lab instrument was used, >1 mL of the test sample was injected into a sample cell, and the instrument was run for measurement. Data analysis was performed based on the Z-Average (nm), Polydispersity Index (PI) value, and the distribution profiles of the Size Distribution by Intensity/Volume. The results were then documented.

### III. Results

According to the above method, VZV gE-4T set forth in SEQ ID NO: 15 was subjected to a binding reaction with NPM-4C set forth in SEQ ID NO: 18, and the binding rate was determined to be 79.2% by SDS-PAGE gray scale method. As shown in FIG. 2, panel a in FIG. 2 showed the results of SDS-PAGE analysis of the prepared VZV gE-NPM disclosed herein, panel b in FIG. 2 showed the results of Western Blot detection of VZV gE-NPM disclosed herein, and panel c in FIG. 2 showed the binding and stability results of VZV gE-NPM disclosed herein. In the SEC identification results in panel c in FIG. 2, peak 1 was the nanoparticles formed after the 6:1 mixing and binding of VZV gE and NPM, and peak 2 was the remaining VZV gE antigen protein after the binding. Panel a in FIG. 3 showed the results of the purification and separation after the binding of VZV gE and NPM, and panel b in FIG. 3 showed the particle size analysis results of naked NPM particles. The DLS results showed that the particle diameter was 27.6 nm, and the product was stable with qualified endotoxin level. B2-B6 shown in panel a in FIG. 3 were the samples contained in and collected from peak 1 of panel c in FIG. 2, and B8-C1 were the samples included in and collected from peak 2 of panel c in FIG. 2. Panel c in FIG. 3 showed the particle size analysis results of gE-NPM binding. The DLS results showed that the particle diameter was 34.4 nm, and the product was stable with qualified endotoxin level.

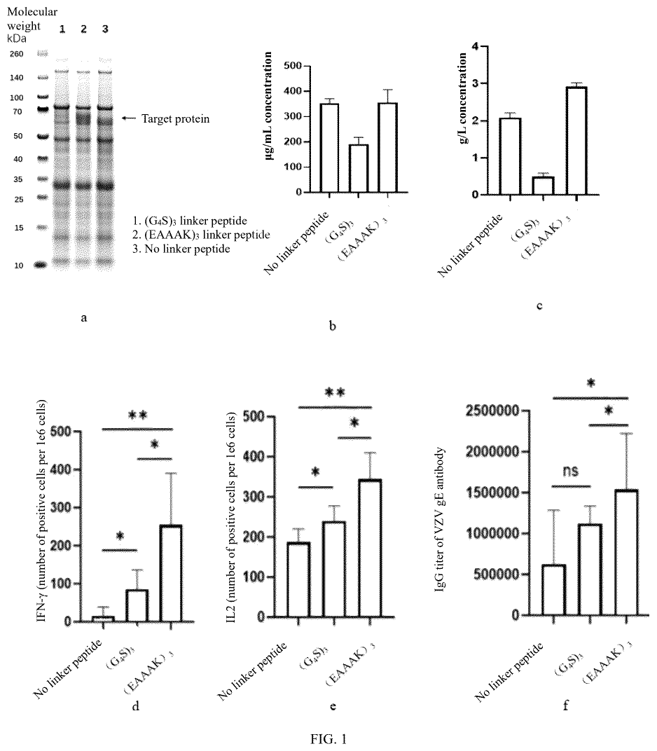

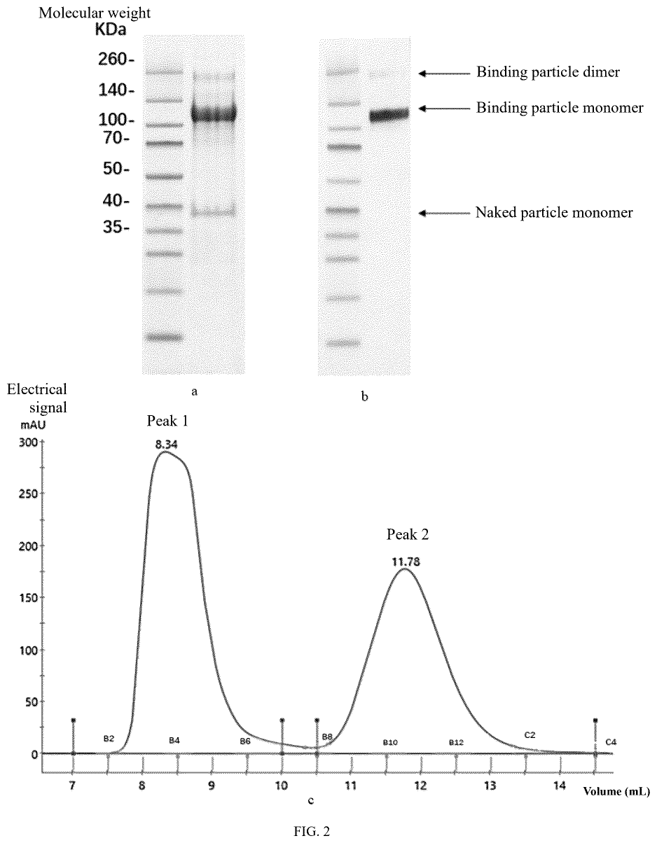

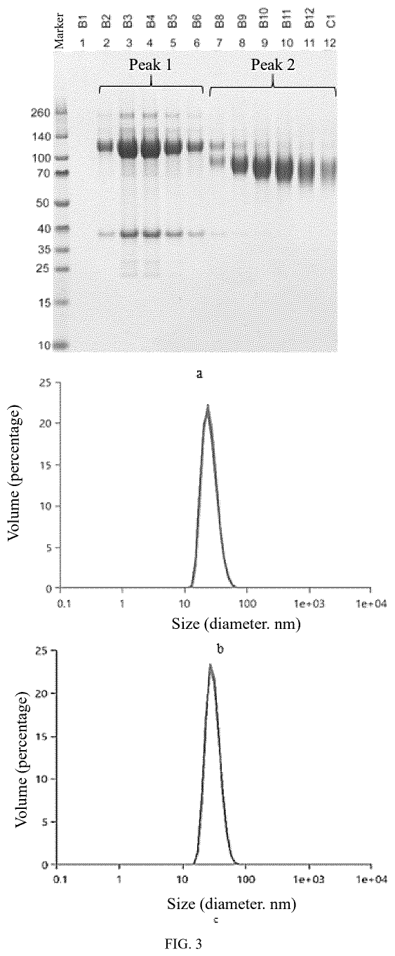

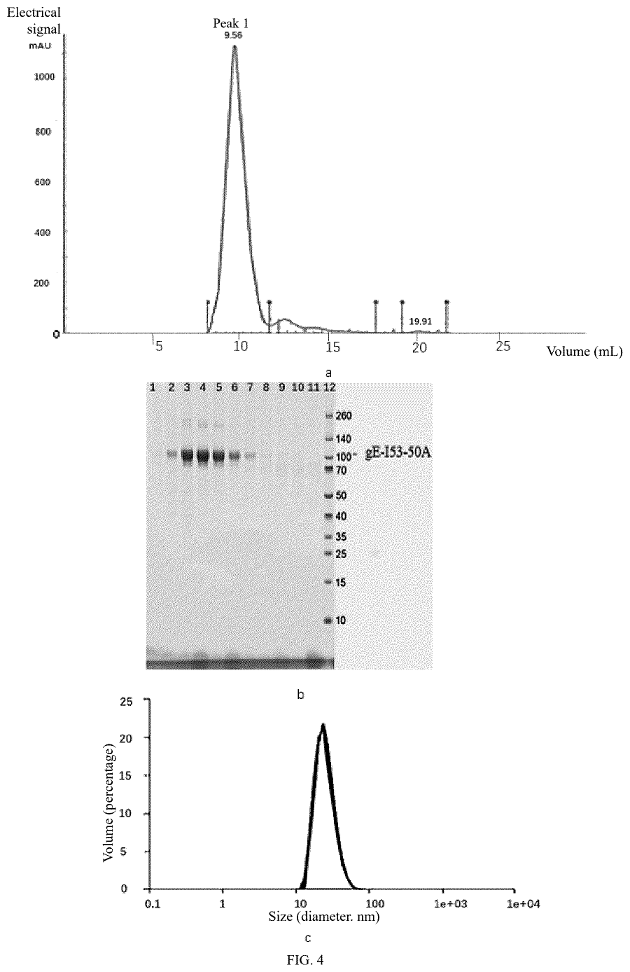

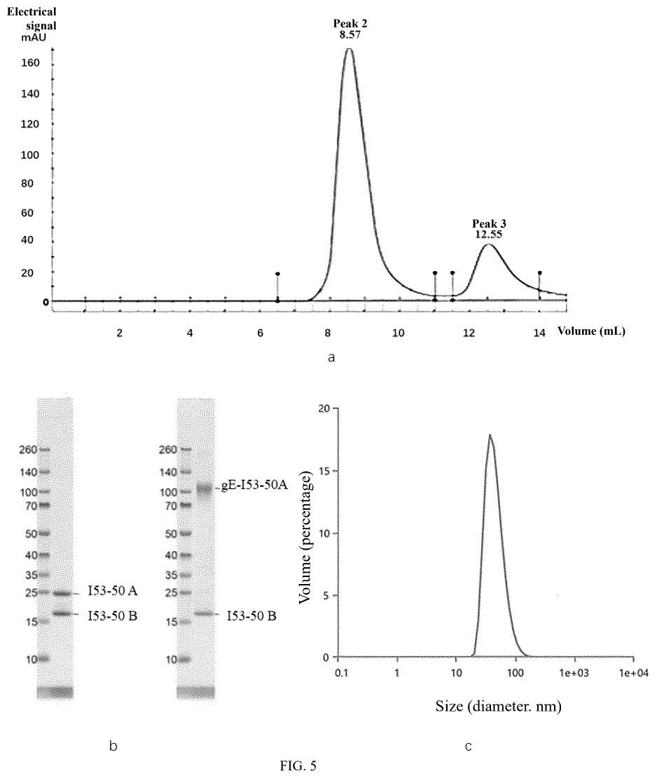

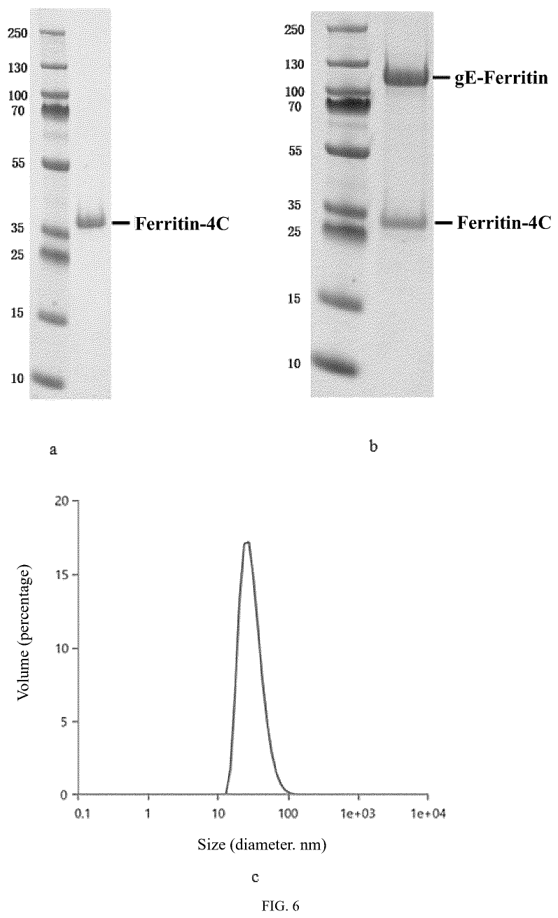

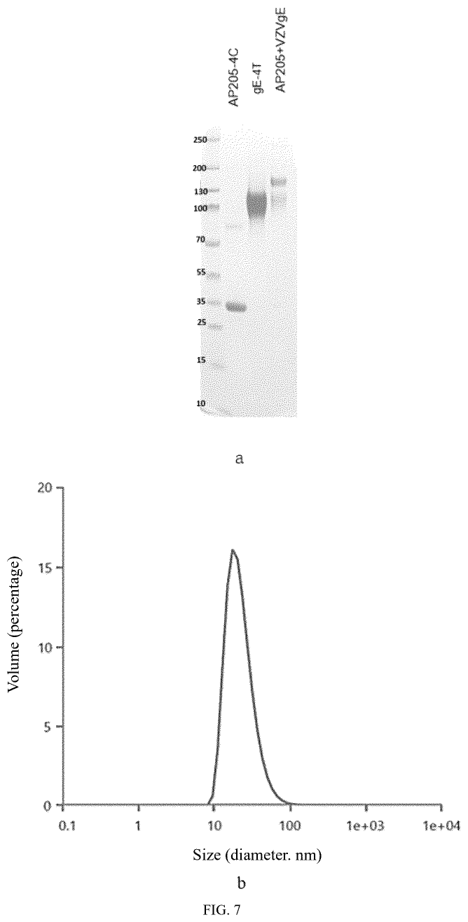

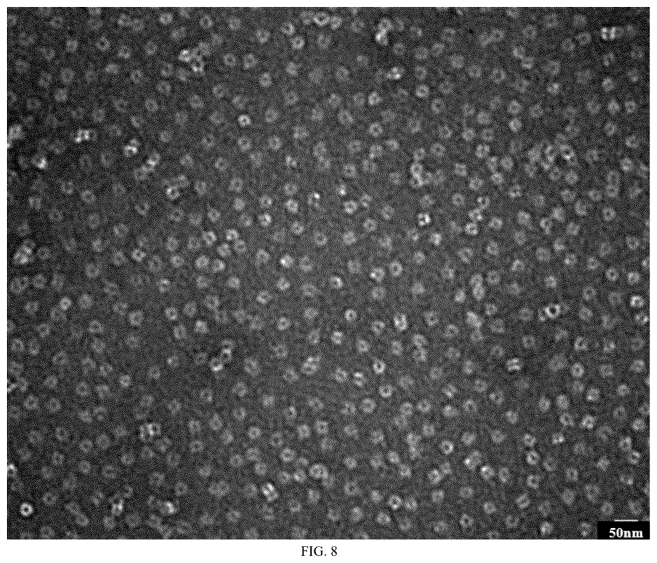

FIG. 8 was an image showing electron microscope examination results of VZV gE-NPM particles (VZV gE-NPM 0.13 mg/mL, 18,500×), which showed that the particles were uniformly distributed without aggregation.

Binding reaction of VZV gE-4T set forth in SEQ ID NO: 16 with NPM-4C achieved the same technical effects as the above results.

The above results indicate that the VZV gE and NPM provided herein were assembled normally, and the molecular weight interval was reasonable.

### Example 6: Expression, Purification, and Binding of VZV gE-I53-50A and I53-50B, and Particle Characterization

Different from other particles, the binding of VZV gE and I53-50 was achieved by expressing and purifying the VZV gE-I53-50A fusion protein and I53-50B separately and allowing them to bind, thereby obtaining VZV gE-I53-50. The sequence structures of I53-50A, I53-50B, and VZV gE-I53-50A in the examples of the present application are shown in Table 9.

### I. Expression and Purification of I53-50B

### 1. Experimental Materials

Capsule filter (Bricap COT: 180 cm²) purchased from Cobetter, membrane cassette purchased from Millipore, Ni Bestarose FF purchased from Bestchrom, and SEC column (HiLoad 16/600 Superdex 200 μg) purchased from Cytiva.

### 2. Experimental Method

1) Expression induction: The encoding gene of I53-50B was expressed in E. coli, and then I53-50B BL21 Condon-plus/BL21(DE3) monoclonal strain was inoculated into 50 mL of LB (Kan+) culture medium, followed by culturing at 37° C. and 250 rpm for 4-5 h. When the OD600 of the culture solution was about 0.6-0.8, the bacterial solution was transferred to an 18° C. environment, IPTG was added until its final concentration was 0.5 mM, and the protein expression was induced at 200 rpm for 16 h.

2) Centrifugation for bacterial cell collection: The cultured bacterial solution was transferred to a labeled clean 50 mL centrifuge tube and centrifuged at room temperature at 6000 rpm to collect the bacterial cells, the culture medium was discarded, the bacterial cells were resuspended in 10 mL of a solution containing 300 mM NaCl, 50 mM Tris 7.4, 1 mM DTT, and 0.75% CHAPS, and the suspension was thoroughly mixed in a vortex mixer.

3) Ultrasonic disruption: A 50 mL tube containing the resuspended bacterial solution was placed in ice for ultrasonic disruption. The samples were sonicated using a No. 2 ultrasonic horn at 100% power, with a cycle of 5 s on and 5 s off. The total sonication duration for each sample was 10 min. 4) Collection of target protein by centrifugation: The centrifugation was performed at 15,000 rpm and 4° C. for 30 min, and the cell lysate supernatant was collected. The sequence of the target protein I53-50B was set forth in SEQ ID NO: 20.

5) Purification of target protein by Histrap: The wash buffer was a solution containing 300 mM NaCl, 50 mM Tris pH 7.4, 1 mM DTT, 0.75% CHAPS, and 30 mM imidazole, and the elution buffer was a solution containing 300 mM NaCl, 50 mM Tris pH 7.4, 1 mM DTT, 0.75% CHAPS, and 300 mM imidazole. Purification was performed using a Histrap Excel-5 mL or Histrap Bogelong-10 mL column. The column was equilibrated with 5 CV of wash buffer, and the target protein was filtered through a 0.22 μm filter, diluted to 45 mL with wash buffer, and then injected from S1. The protein impurities was washed off with 10 CV of wash buffer, and the target protein was eluted with 5 CV of elution buffer.

6) Buffer exchange: The target protein solution eluted with Histrap was buffer-exchanged into a solution containing 300 mM NaCl, 50 mM Tris pH 7.4, and 0.75% CHAPS using a concentration tube, and then the protein concentration was determined. The target protein solution was stored at an appropriate temperature for the subsequent binding reaction.

### II. Expression, Purification, and Binding Reaction of VZV gE-I53-50A Target Protein

### 1. Experimental Materials

capsule filter (Bricap COT: 180 cm²) purchased from Cobetter, membrane cassette purchased from Millipore, Ni Bestarose FF purchased from Bestchrom, and SEC column (HiLoad 16/600 Superdex 200 μg) purchased from Cytiva

### 2. Experimental Method

1) The encoding gene of VZV gE-I53-50A was inserted into a eukaryotic cell expression vector pcDNA3.4 for expression in CHO-S cells. The CHO-S cells were thawed and cultured in suspension in an ExpiCHO culture medium. The culture conditions are as follows: temperature: 37° C.; humidity: 80%; CO₂ concentration: 8%; rotation speed: 120 rpm.

2) CHO-S cells were expanded, with a doubling time of about 16 h/generation. When the density reached 6×10⁶ cells/mL, transfection was ready to start.

3) The plasmid carrying the VZV gE target gene was diluted with cold OptiPRO™ Medium (4° C.).

4) The ExpiFectamine™ CHO Reagent was inverted 4-5 times before use to ensure thorough mixing. The ExpiFectamine™ CHO Reagent was then diluted with cold OptiPRO™ Medium (4° C.), and the mixture was immediately mixed with the diluted DNA after being left to stand for 2-3 min.

5) The mixed solution obtained in steps 3 and 4 was left to stand for 2-3 min (no more than 5 min) and then added to the CHO-S cells prepared in advance. The transfection mixture was added while the cells were continuously shaken to allow the transfection complex to be fully mixed with the cells.

6) After transfection, the cells were cultured for expression in a shaker at 37° C., 80% humidity, 8% CO₂, and a rotation speed of 120 rpm.

7) On day 1 of expression, the ExpiCHO™ Enhancer was added as a supplement, the Max Titer expression mode was selected, and then the ExpiCHO™ Feed solution was added as a supplement.

The solutions were slowly added with continuous shaking of the culture flask. Subsequently, the culture temperature was reduced to 32° C.

8) On day 5 of expression, the ExpiCHO™ Feed solution was added, as a supplement, slowly under the Max Titer expression mode with continuous shaking of the culture flask.

9) The cell viability and cell number were recorded on days 5/7/9/11 of expression as appropriate, and samples were taken on day 7 for SDS-PAGE analysis.

10) Collection of cell expression supernatant: The supernatant was collected by centrifugation at 8000 rpm for 30 min, filtered through a 0.22 μm filter membrane, and marked and stored.

11) The cell supernatant was 3-fold diluted with equilibration buffer (20 mM PBS, pH 6.0) to reduce the conductance, and the pH was adjusted to 6.0 with phosphoric acid. VZV gE-I53-50A was purified by a DEAE chromatography column. The equilibration buffer was 20 mM PBS (pH 6.0), and the elution buffer was 20 mM PBS (pH 6.0) containing 1 M NaCl.

12) VZV gE-I53-50A was purified again by SEC column, as shown in panel a in FIG. 4. The elution buffer was a solution containing 20 mM Tris-HCl and 150 mM NaCl (pH 7.4). The purified gE-I53-50A was identified by SDS-PAGE, as shown in panel b in FIG. 4. The VZV gE-I53-50A protein concentration was determined and the protein was cryopreserved at −80° C. The target protein sequence VZV gE-I53-50A was set forth in SEQ ID NO: 21.

13) VZV gE-I53-50A and I53-50B were mixed in a mass ratio of 1:3 for a binding assay under the conditions of pH 7.4, 20 mM Tris-HCl, 150 mM NaCl, and reaction at the room temperature of 25° C. for 2 h. The binding product VZV gE-I53-50 was then obtained.

14) The binding product was separated and purified by using an SEC column, as shown in panel a in FIG. 5. The purified VZV gE-I53-50 was identified by SDS-PAGE and compared with I53-50A and I53-50B, as shown in panel b in FIG. 5. The particle size of VZV gE-I53-50 was measured and compared with that of I53-50, as shown in panel c in FIG. 4 and panel c in FIG. 5.

### III. Particle Characterization

1. Experimental materials: VZV gE-I53-50 particles prepared in the above example
2. Assay method: same as in Example 5

### IV. Results

Panel a in FIG. 4 to panel c in FIG. 5 showed the results of binding and stability of the herpes zoster VZV gE-I53-50 particles claimed by the present disclosure: panel a in FIG. 4 and panel b in FIG. 4 showed the results of preparation and purification of VZV gE-I53-50A antigen, where peak 1 of panel a in FIG. 4 was the result of purified VZV gE-I53-50A, and lanes 2 to 8 of panel b in FIG. 4 were the samples contained in and collected from peak 1 of panel a in FIG. 4. The DLS results in panel c in FIG. 4 showed that the I53-50 particles had a diameter of 30.7 nm, and the product was stable with qualified endotoxin level. Panels a and b in FIG. 5 showed the results of the binding of VZV gE-I53-50 claimed by the present disclosure and the purification after binding. The peak 2 of panel a in FIG. 5 was the nanoparticles formed after the 1:3 mixing and binding of VZV gE-I53-50A and I53-50B, and the peak 3 was the remaining I53-50B protein after the binding. Panel b in FIG. 5 showed the analytical identification results of VZV gE-I53-50A after purification and its comparison with I53-50A and I53-50B in SDS-PAGE; the DLS results of panel c in FIG. 5 showed that VZV gE-I53-50 particles had a diameter of 60.15 nm, and the product was stable with qualified endotoxin level.

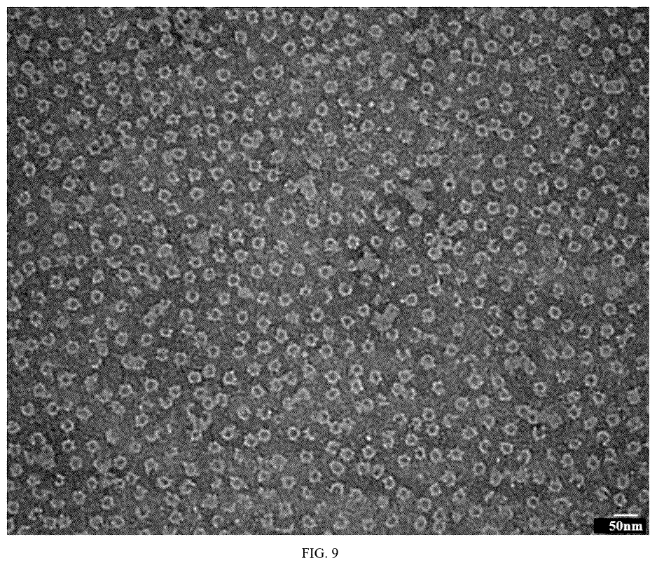

The electron microscope examination results of gE-I53-50 particles are shown in FIG. 9. The image showing the electron microscope examination results of VZV gE-I53-50 particles (VZV gE-I53-50 0.13 mg/mL, 18,500×) showed that VZV gE-I53-50A was able to react with I53-50B to form nanoparticles, and the molecular weight was in line with the expected value. The image showed that the particles were uniformly distributed without aggregation.

TABLE 9
153-50A, 153-50B, and VZV gE-153-50A in
the examples of the present application
Sequence

I53-50A:
MKMEELFKKHKIVAVLRANSVEEAIEKAVAVFAGGVHLIEITFTV
PDADTVIKALSVLKEKGAIIGAGTVTSVEQCRKAVESGAEFIVSP
HLDEEISQFCKEKGVFYMPGVMTPTELVKAMKLGHTILKLFPGEV
VGPQFVKAMKGPFPNVKFVPTGGVNLDNVCEWFKAGVLAVGVGSA
LVKGTPDEVREKAKAFVEKIRGCTE
(SEQ ID NO: 19)
I53-50B:
MNQHSHKDHETVRIAVVRARWHAEIVDACVSAFERRITINAAMRD
IGGDRFAVDVFDVPGAYEIPLHARTLAETGRYGAVLGTAFVVNGG
IYRHEFVASAVINGMMNVQLNTGVPVLSAVLTPHNYDKSKAHTLL
FLALFAVKGMEAARACVEILAAREKIAAGSGHHHHHH
(SEQ ID NO: 20)
VZV gE-153-50A:
MEFGLSWVFLVAIIKGVQCSVLRYDDFHIDEDKLDTNSVYEPYYH
SDHAESSWVNRGESSRKAYDHNSPYIWPRNDYDGFLENAHEHHGV
YNQGRGIDSGERLMQPTQMSAQEDLGDDTGIHVIPTLNGDDRHK
IVNVDQRQYGDVFKGDLNPKPQGQRLIEVSVEENHPFTLRAPIQR
IYGVRYTETWSFLPSLTCTGDAAPAIQHICLKHTTCFQDVVVDVD
CAENTKEDQLAEISYRFQGKKEADQPWIVVNTSTLFDELELDPPE
IEPGVLKVLRTEKQYLGVYIWNMRGSDGTSTYATFLVTWKGDEKT
RNPTPAVTPQPRGAEFHMWNYHSHVFSVGDTFSLAMHLQYKIHEA
PFDLLLEWLYVPIDPTCQPMRLYSTCLYHPNAPQCLSHMNSGCTF
TSPHLAQRVASTVYQNCEHADNYTAYCLGISHMEPSFGLILHDGG
TTLKFVDTPESLSGLYVFVVYFNGHVEAVAYTVVSTVDHFVNAIE
ERGFPPTAGQPPATTKPKEITPVNPGTSPLIRYAAWTGGGGSGGS
GSEKAAKAEEAARKMEELFKKHKIVAVLRANSVEEAIEKAVAVFA
GGVHLIEITFTVPDADTVIKALSVLKEKGAIIGAGTVTSVEQCRK
AVESGAEFIVSPHLDEEISQFCKEKGVFYMPGVMTPTELVKAMKL
GHTILKLFPGEVVGPQFVKAMKGPFPNVKFVPTGGVNLDNVCEWF
KAGVLAVGVGSALVKGTPDEVREKAKAFVEKIRGCTEGGSHHHHH
H
(SEQ ID NO: 21)

### Example 7 Expression, Purification, and Binding of VZV gE and Ferritin, and Particle Characterization

The recombinant nanoparticle protein Ferritin was fused, at its N-terminus, with the binding peptide 2 set forth in SEQ ID NO: 2 via the linker peptide 2 set forth in SEQ ID NO: 8 to form a fusion protein, which was binding peptide 2-Ferritin fusion protein, i.e., “Ferritin-4C”. The sequences of Ferritin and Ferritin-4C in the examples of the present application are shown in Table 10.

TABLE 10
Ferritin and Ferritin-4C in the examples
of the present application

| Polypeptide | |
|---|---|
| name | Sequence |
| Ferritin | DIIKLLNEQVNKEMNSSNLYMSMSSWCYTH |
| | SLDGAGLFLFDHAAEEYEHAKKLIIFLNEN |
| | NVPVQLTSISAPEHKFEGLTQIFQKAYEHE |
| | QHISESINNIVDHAIKSKDHATFNFLQWYV |
| | AEQHEEEVLFKDILDKIELIGNENHGLYLA |
| | DQYVKGIAKSRKS |
| | (SEQ ID NO: 22) |
| Ferritin-4C | MHHHHHHGGSDSATHIKFSKRDEDGKELAG |
| | ATMELRDSSGKTISTWISDGQVKDFYLYPG |
| | KYTFVETAAPDGYEVATAITFTVNEQGQVT |
| | VNGKATKGDAHISGGSGGDIIKLLNEQVNK |
| | EMNSSNLYMSMSSWCYTHSLDGAGLFLFDH |
| | AAEEYEHAKKLIIFLNENNVPVQLTSISAP |
| | EHKFEGLTQIFQKAYEHEQHISESINNIVD |
| | HAIKSKDHATFNFLQWYVAEQHEEEVLFKD |
| | ILDKIELIGNENHGLYLADQYVKGIAKSRK |
| | S |
| | (SEQ ID NO: 23) |

### I. Expression and Purification of Ferritin-4C

### 1. Experimental Materials

Capsule filter (Bricap C01: 180 cm²) purchased from Cobetter, membrane cassette purchased from Millipore, HisTrap excel purchased from Cytiva, and SEC column (HiLoad 16/600 Superdex 200 μg) purchased from Cytiva.

### 2. Experimental Method

1) Expression induction conditions: The encoding gene of Ferritin-4C was expressed in E. coli, and then Ferritin-4C BL21(DE3) monoclonal strain was inoculated into 400 mL of an LB (Amp⁺) culture medium, followed by culturing at 37° C. and 220 rpm for 4-5 h. When the OD600 of the culture solution was about 0.6-0.8, the bacterial solution was transferred to an 18° C. environment, IPTG was added until its final concentration was 0.5 mM, and the protein expression was induced at 200 rpm for 16 h. The sequence of the target protein Ferritin-4C was set forth in SEQ ID NO: 23.

2) Centrifugation for bacterial cell collection: The cultured bacterial solution was centrifuged at 7000 g and room temperature to collect the bacterial cells, the culture medium was discarded, and the bacterial cells were resuspended in 40 mL of a solution containing 150 mM NaCl, 20 mM Tris pH 7.4.

3) Ultrasonic disruption: The resuspended bacterial solution was placed in an ice-water bath for ultrasonic disruption. The samples were sonicated using a No. 2 ultrasonic horn at 50% power, with a cycle of 3 s on and 7 s off. The total sonication duration was 12 min.

4) Collection of target protein by centrifugation: The centrifugation was performed at 13,000 g and 4° C. for 30 min. The supernatant was discarded, and the inclusion bodies were dissolved in a solution containing 20 mM Tris-HCl, 150 mM NaCl, 8 M Urea, and 2% Triton X-100 pH 7.4 for 1 h.

5) Purification of target protein by Histrap: The wash buffer 1 was 20 mM Tris-HCl, 150 mM NaCl, 8 M urea, pH 7.4, the wash buffer 2 was 20 mM Tris-HCl, 150 mM NaCl, 8 M urea, 2% Triton X-100, pH 7.4, and the elution buffer was 20 mM Tris-HCl, 150 mM NaCl, 1 M imidazole, pH 7.4. After the inclusion bodies were dissolved, the mixture was centrifuged at 13,000 g for 30 min, and the supernatant was taken and loaded onto a Histrap excel-5 mL NI column. The Histrap Excel-5 mL column was equilibrated with 10 CV of wash buffer 2, followed by sample loading. After loading, the chromatography column was washed with 40 CV of wash buffer 2 to remove endotoxins, followed by washing with 10 CV of wash buffer 1 to eliminate Triton X-100. Subsequently, 10 CV of a 2% elution buffer was applied to wash away protein impurities. Target protein was then eluted using 15 CV of elution buffer by linear gradient elution from 2% to 100%. After the elution, the purity of the eluted protein was analyzed by SDS-PAGE.

6) Dilution refolding: The buffer for both dilution refolding and SEC was 20 mM Tris-HCl, 150 mM NaCl, 25% (w/v) sucrose, pH 7.4. The target protein purified by a nickel column was concentrated using a concentration tube. During the concentration process, a gradient dilution with SEC buffer was applied to reduce the urea concentration to 0.25 M. The sample was concentrated to 1 mL and subsequently subjected to separation and purification by an SEC column. The elution buffer was the SEC buffer. Following elution, the protein purity was determined by SDS-PAGE. The SDS-PAGE analysis of the purified Ferritin-4C is shown in panel a in FIG. 6. The protein concentration was determined by the BCA method, and the protein was stored at an appropriate temperature for subsequent binding reactions.

### II. Binding of VZV gE-Binding Peptide 1 and Binding Peptide 2-Ferritin and Purification of Binding Product

The VZV gE-binding peptide 1 set forth in SEQ ID NO: 15 or SEQ ID NO: 16 and the binding peptide 2-Ferritin (Ferritin-4C) were mixed at ratio of 6:1 based on their BCA protein concentrations, and a 50% (w/v) sucrose stock solution was added until a final sucrose concentration of about 25% (w/v) was achieved. A 1 M Tris-HCl pH 7.4 stock solution equivalent to 10% of the total reaction volume was added to stabilize the pH. The binding reaction was carried out at 22° C. for 48 h.

The binding rate for the above VZV gE-binding peptide 1 and the binding peptide 2-Ferritin was calculated to be 80% by SDS-PAGE gray scale method.

The binding product was separated and purified by an SEC column (purification buffer: 20 mM Tris-HCl, 150 mM NaCl, 25% (w/v) sucrose, pH 7.4), and the VZV gE-Ferritin component was collected. The purified VZV gE-Ferritin nanoparticles were identified by SDS-PAGE, as shown in panel b in FIG. 6.

### III. Particle Characterization

1. Experimental material: VZV gE-Ferritin particles prepared in the above example.
2. Assay method: same as in Example 5.

### IV. Results

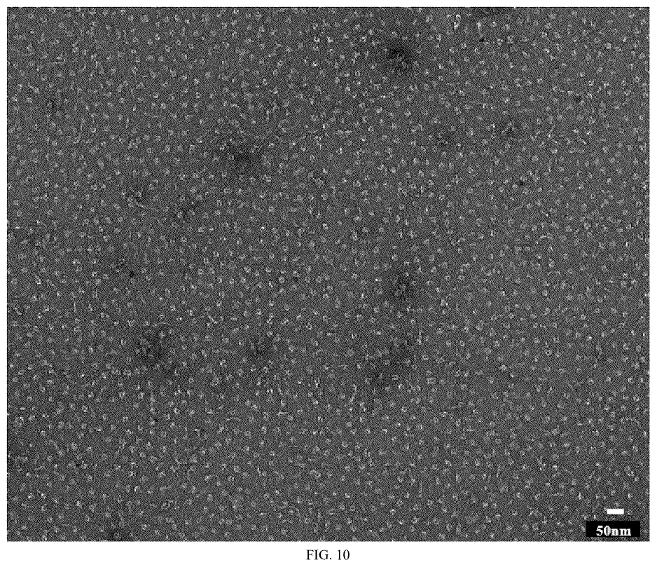

Panel a in FIG. 6 showed SDS-PAGE identification results of purified Ferritin-4C nanoparticles claimed by the present disclosure, panel b in FIG. 6 showed SDS-PAGE identification results of varicella-zoster nanoparticle VZV gE-Ferritin claimed by the present disclosure after binding and purification, panel c in FIG. 6 showed the particle size measurement results of VZV gE-Ferritin nanoparticles, and DLS results showed that the particle diameter was 34.17 nm and the product was stable; FIG. 10 was an image showing electron microscope examination results of VZV gE-Ferritin nanoparticles after negative staining, and the image showed that the particles are uniformly distributed without aggregation. The above results indicate that the VZV gE-Ferritin particles provided herein were assembled normally and the molecular weight interval was reasonable. FIGS. 6 and 10 showed the effects obtained by the binding reaction of VZV gE-4T set forth in SEQ ID NO: 15 with Ferritin-4C. When VZV gE-4T set forth in SEQ ID NO: 16 and Ferritin-4C were subjected to the binding reaction, the same technical effects as the above results were obtained.

### Example 8: Expression and Purification of AP205 Fusion Protein, Binding to VZV gE, and Particle Characterization

The recombinant nanoparticle protein AP205 was fused, at its N-terminus, with the binding peptide 2 set forth in SEQ ID NO: 2 via the linker peptide 2 set forth in SEQ ID NO: 9 to form a fusion protein, which was binding peptide 2-AP205 fusion protein, i.e., “AP205-4C”. The sequences of AP205 and AP205-4C in the examples of the present application are shown in Table 11.

TABLE 11
Sequences of AP205 and AP205-4C in the
examples of the present application

| Polypeptide | |
|---|---|
| name | Sequence |
| AP205 | ANKPMQPITSTANKIVWSDPTRLSTTFSAS |
| | LLRQRVKVGIAELNNVSGQYVSVYKRPAPK |
| | PEGCADACVIMPNENQSIRTVISGSAENLA |
| | TLKAEWETHKRNVDTLFASGNAGLGFLDPT |
| | AAIVSSDTTA |
| | (SEQ ID NO: 24) |
| AP205-4C | MGSSHHHHHHGSGDSATHIKFSKRDEDGKE |
| | LAGATMELRDSSGKTISTWISDGQVKDFYL |
| | YPGKYTFVETAAPDGYEVATAITFTVNEQG |
| | QVTVNGKATKGDAHIGSGGSGGSGANKPMQ |
| | PITSTANKIVWSDPTRLSTTFSASLLRQRV |
| | KVGIAELNNVSGQYVSVYKRPAPKPEGCAD |
| | ACVIMPNENQSIRTVISGSAENLATLKAEW |
| | ETHKRNVDTLFASGNAGLGFLDPTAAIVSS |
| | DTTA |
| | (SEQ ID NO: 25) |

### I. Expression and Purification of AP205-4C

### 1. Experimental Materials

Ni-NTA purchased from QIAGEN, nuclease (Benzonase) purchased from Sigma, and dialysis bag (300 KD) from Spectrum Labs.

### 2. Experimental Method

The encoding gene of AP205-4C was expressed in E. coli, and the bacterial sludge (from a 2.4 L culture) was resuspended in 50 mL of dissolution buffer, incubated at room temperature for 15 min, and then incubated in a ice bath for 10 min. The suspension was then subjected to sonication for 12 cycles (30 s per cycle with 60 s intervals between cycles) at a 30% ultrasonic power ratio. The ultrasonic lysate was centrifuged for 20 min (15,000 g, 4° C.), and the supernatant was discarded. After the pellet was collected, the inclusion bodies were resuspended in a urea buffer and stirred at 300 rpm at room temperature overnight. The next day, the urea resuspension was centrifuged for 80 min (15,000 g, 25° C.), the supernatant was collected and filtered with a syringe (0.45 μm), and 250 U of nuclease (Benzonase, Sigma) was added, followed by incubation at room temperature for 5 min. The sequence of AP205-4C was set forth in SEQ ID NO: 25.

The Ni-NTA (QIAGEN) column packing (3 mL) was washed twice with ultrapure water (5 CV, 15 mL) and twice with the equilibration buffer 2 (5 CV, 15 mL), and the sample resuspension was added, followed by vibration at room temperature for 10 min and centrifugation at 4700 g for 4 min. The supernatant was removed. The column packing was diluted with 45 mL of equilibration buffer 1 for refolding, and the column packing was left to stand, followed by removal of the supernatant. This operation was repeated 3 times. The column packing was washed with 45 mL of rinsing buffer 1 and left to stand, and the supernatant was removed. This operation was repeated 3 times. The column packing was washed with 45 mL of rinsing buffer 2 and left to stand, and the supernatant was removed. This operation was repeated 6 times in total. The column packing was washed with 15 mL of rinsing buffer 3 and left to stand, and the supernatant was removed. This operation was repeated 3 times. The column packing was washed with 1 mL of elution buffer, followed by vibration at room temperature (900 rpm) for 10 min and centrifugation for 10 min (13,000 g, 4° C.), and the supernatant eluate was taken for later use. 1 mL of the eluate was placed in a dialysis bag (300 kD, Spectrumlabs), sealed, and dialyzed against 1 L of dialysis buffer for 3 h, followed by dialysis against 1 L of fresh dialysis buffer overnight. The next day, the eluate was taken out, centrifuged, and stored at 4° C. for later use. See Table 12 below for specific parameters.

TABLE 12
Chromatography method

| Chromatography | | | |
|---|---|---|---|
| procedures | Chromatography buffer/condition | Parameter | |
| Dissolution buffer | 20 mM Tris-HCl, 0.1% (v/v) Tween 20, 0.1% (v/v) TritonX-100, 150 mM | 50 | ml |
| | NaCl, pH 7.8 | | |
| Urea buffer | 20 mM Tris-HCl, 8M Urea, 0.1% (v/v) Tween 20, 0.1% (v/v) TritonX-100, | 50 | ml |
| | 150 mM NaCl, pH 7.8 | | |
| Equilibration | 20 mM Tris-HCl, 0.1% (v/v) Tween 20, 0.1% (v/v) TritonX-100, | 45 | CV |
| buffer 1 | 150 mM NaCl, pH 7.8 | | |
| Equilibration | 20 mM Tris-HCl, 8M Urea, 0.1% (v/v) Tween 20,0.1% (v/v) TritonX-100, | 15 | CV |
| buffer 2 | 150 mM NaCl, 75 mM Imidazole, pH 7.8 | | |
| Rinse buffer 1 | 20 mM Tris-HCl, 0.1% (v/v) Tween 20, 0.1% (v/v) TritonX-100, | 45 | CV |
| | 150 mM NaCl, 100 mM Imidazole, pH 7.8 | | |
| Rinse buffer 2 | 20 mM Tris-HCl, 0.1% (v/v) Tween 20, 0.1% (v/v) TritonX-114, | 90 | CV |
| | 150 mM NaCl, 100 mM Imidazole, pH 7.8 | | |
| Rinse buffer 3 | 20 mM Tris-HCl, 0.1% (v/v) Tween 20, 150 mM NaCl, 100 mM | 15 | CV |
| | Imidazole, pH 7.8 | | |
| Elution buffer | 25 mM sodium citrate, 50 mM glycine, 0.1% (v/v) Tween 20, | 1 | ml |
| | 2M imidazole, pH 8.5 | | |
| Dialysis buffer | 25 mM sodium citrate, 50 mM glycine, 0.1% (v/v) Tween 20, | 2 | L |
| | pH 8.0 | | |

### Results and Analysis

Through purity testing, it was found that after refinement through the above purification and dialysis combination, the purity of the resulting product reached 95.0% or more.

### II. Binding of VZV gE-Binding Peptide 1 and Binding Peptide 2-AP205 and Purification of Binding Product

1. Generation of VZV gE-AP205 Binding Product

The VZV gE-4T set forth in SEQ ID NO: 15 or SEQ ID NO: 16 and AP205-4C were mixed at ratio of 2:1 based on their BCA protein concentrations, and a 50% (w/v) sucrose stock solution was added until a final sucrose concentration of about 25% (w/v) was achieved. A pH 6.2 stock solution containing 200 mM sodium citrate (Na₃C₆H₅O₇·2H₂O) and 40 MM Na₂HPO₄ was added to stabilize the pH. The binding reaction was carried out at 22° C. for 24 h.

2. Purification of VZV gE-AP205 Binding Product:

The VZV gE-AP205 binding product was purified using a dialysis bag (300 kD, Spectrumlabs), and the gE antigen unbound to AP205-4C was separated and removed using a magnetic stirrer (350 rpm). The dialysis was performed 4 times for not less than 4 h each, and the dialysis buffer was 50 mM glycine, 25 mM sodium citrate, 0.1% (v/v) Tween 20, pH 6.2. After dialysis, the binding product was taken out and centrifuged (13,000 g, 4° C.) for 10 min, and the supernatant was collected and stored at 4° C.

### III. Particle Characterization

1. Experimental material: VZV gE-AP205 particles prepared in the above example.
2. Assay method: same as in Example 5.

### IV. Results

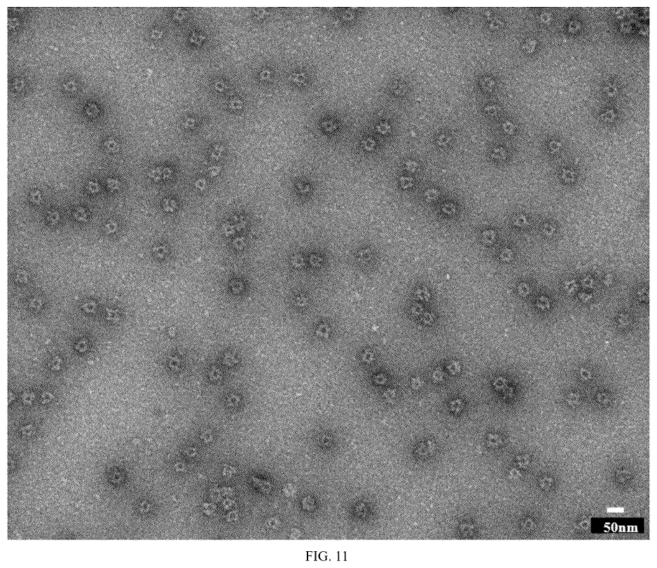

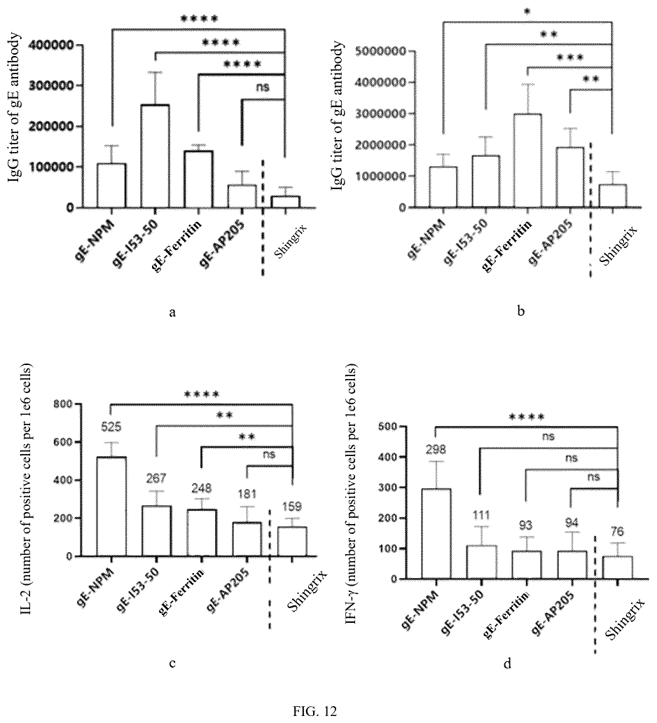

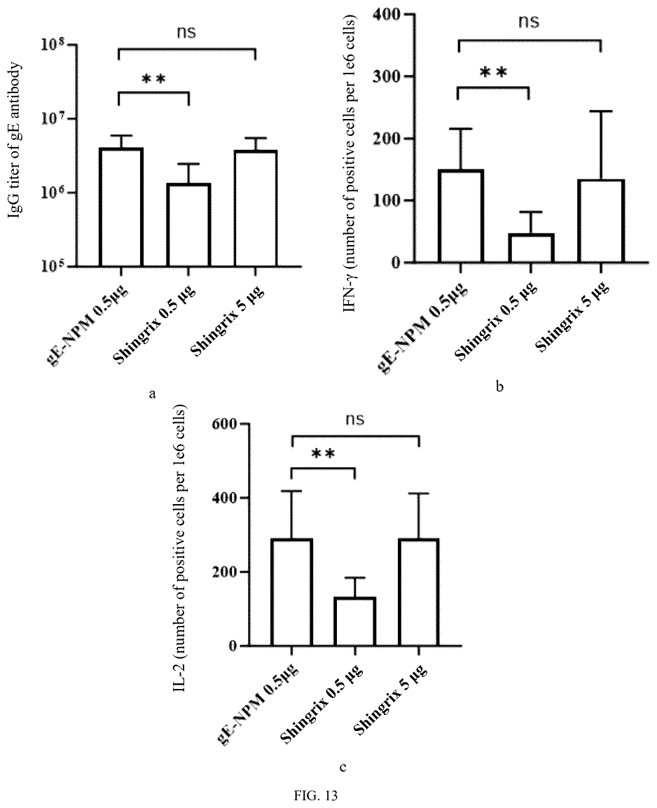

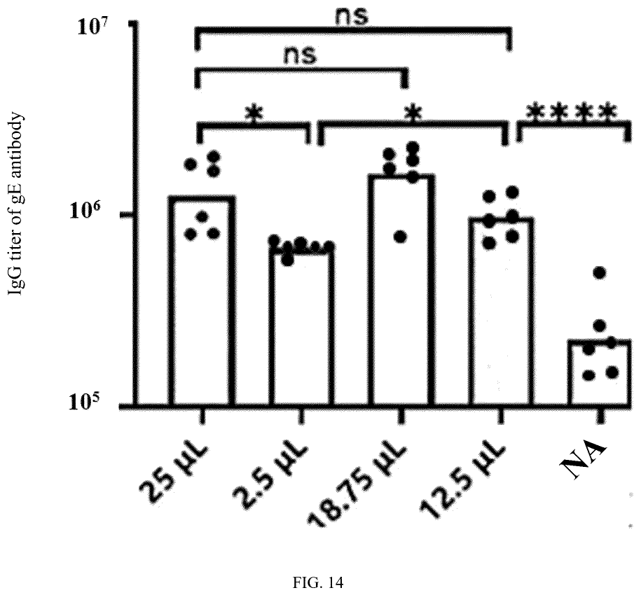

As shown in FIG. 7, panel a in FIG. 7 showed the SDS-PAGE analysis results of the prepared VZV gE-AP205 claimed by the present disclosure. Panel b in FIG. 7 showed the DLS measurement results of AP205-4C, and the results showed that the particle diameter was 17.31 nm, and the endotoxin level was qualified. FIG. 11 was an image showing electron microscope examination results of VZV gE-AP205 nanoparticles after negative staining, which showed that the particles were uniformly distributed without aggregation.

FIGS. 7 and 11 showed the effect obtained by the binding reaction of VZV gE-4T set forth in SEQ ID NO: 15 with AP205-4C. When VZV gE-4T set forth in SEQ ID NO: 16 and AP205-4C were subjected to the binding reaction, the same technical effects as the above results were obtained.

The above results indicate that the VZV-gE and AP205 provided herein were assembled normally, and the molecular weight interval was reasonable.

### SUMMARY

It can be seen from Examples 5-8 that the four nanoparticle products prepared can all be used as the varicella-zoster nanoparticle immunogenic complex claimed by the present disclosure, and can all achieve relatively ideal technical effects, including a uniform particle size, a homogeneous and aggregation-free distribution, and compliant endotoxin levels, all of which demonstrate that they are suitable for non-clinical development and antibody immunogenicity assay and thereby are suitable for use as varicella-zoster nanoparticle candidate vaccines.

### Example 9: Particle Thermostability

### I. Experimental Method

1. Experimental instrument: UNcle all-in-one protein stability analyzer
2. Procedures: Protein samples at the same concentration (0.1 mg/mL) were added to a UNi tube at 9 μL/well, with 3 replicate wells set for each sample. Within the temperature range of 25-95° C., the temperature ramping rate was set at 1° C./min, and the Tm and Tagg266 values were determined for each protein 3 times. The trend of BCM was analyzed to obtain the results.

### II. Experimental Results

The thermostability of the four particles VZV gE-NPM (formed by VZV gE-4T set forth in SEQ ID NO: 15 and NPM-4C set forth in SEQ ID NO: 18), VZV gE-I53-50, VZV gE-Ferritin, and VZV gE-AP205 obtained in the above examples was determined: VZV gE-NPM, VZV gE-I53-50, and VZV gE-AP205 had Tagg266 (° C.) of 61.5±4.88, 53.1±6.02, and 82.3±2.50, respectively, as measured by SLS at a wavelength of 266 nm using UNcle (the Tagg266 (° C.) of gE-Ferritin was unmeasurable using the Uncle instrument). The measured Tm values (° C.) of VZV gE-I53-50, VZV gE-Ferritin, and VZV gE-AP205 were 59.0±0, 60.3±0.45, and 69.7±0.95, respectively (the Tm (° C.) of gE-NPM was unmeasurable using the Uncle instrument). The VZV gE-NPM formed by VZV gE-4T set forth in SEQ ID NO: 16 and NPM-4C set forth in SEQ ID NO: 18 achieved the same technical effects as the above results.

The above data show that the four particles have good thermostability.

### Example 10: Immunogenicity Test of VZV gE-NPM, VZV gE-I53-50, VZV gE-Ferritin, and VZV gE-AP205 in Balb/c Mice

Following the successful preparation of the varicella-zoster nanoparticle immunogenic complexes provided herein, the above compositions were evaluated as vaccines in various non-clinical cellular and antibody immunogenicity tests, with the commercially available varicella-zoster recombinant protein vaccine Shingrix® of GSK serving as the reference.

### 1. Experimental Materials

### (1) Experimental Animals

The recombinant varicella-zoster vaccine (Shingrix®) was purchased from GSK, and female SPF-grade Balb/c mice aged 5-6 weeks were purchased from Vital River. The VZV gE polypeptide was synthesized by Nanjing GenScript. The mice passing the quarantine were marked with metal ear tags, randomly grouped based on the body weight, housed at 4 mice per cage after grouping, and given free access to food and water. The animals were housed in a SPF animal room and provided with sterile feed and sterilized deionized water specialized for SPF animals. The housing environment was maintained on a 12-h light/dark cycle, at a temperature of 21±2° C. and a humidity of 30-70%.

### (2) Test Samples and Control

### 1) Drug Substance of Test Vaccine

The drug substances of the test vaccine proteins were prepared by Guangzhou Patronus Biotech Co., Ltd.: VZV gE-NPM (Examples 4-5), VZV gE-I53-50 (Example 6), VZV gE-Ferritin (Example 7), VZV gE-AP205 (Example 8), and VZV gE-4T (Examples 2-3).

### 2) Test Vaccine Adjuvants

Adjuvant 1: squalene 10.50 mg (4.2%), Span 85 1.25 mg (0.5%), Tween 80 1.25 mg (0.5%), citric acid 0.04 mg (0.264%), sodium citrate 0.66 mg (0.016%) (w/w)

Adjuvant 1 was prepared by Guangzhou Patronus Biotech Co., Ltd. according to the following steps:

Squalene and Span 85 were weighed out and uniformly stirred by a magnetic stirrer to obtain an oil phase. Tween 80 and water for injection were weighed out and stirred by a magnetic stirrer until the Tween 80 was completely dissolved. A sodium citrate buffer was added into water for injection, and the mixture was stirred uniformly. Then, the prepared Tween 80 solution was added, and the mixture was stirred uniformly to obtain an aqueous phase. The dispersion machine was soaked in the aqueous phase. The dispersion machine was turned on, and the oil phase was slowly dripped into the aqueous phase to form a primary emulsion. The primary emulsion was poured into a microfluidizer and homogenized at 12,000 psi until the average particle size was less than 180 nm. The average particle size was about 160 nm. The resulting product was filtered through a 0.2 μm filter membrane and sterilized to obtain the squalene-based adjuvant 1.

### 3) Vaccine Control

Varilrix® (varicella attenuated vaccine), Shingrix® (recombinant varicella-zoster vaccine)

### 2. Experimental Method

### (1) Preparation of Vaccine

### 1) Preparation of Vaccines Disclosed Herein

The drug substance of immunogenic complex (VZV gE-NPM) was diluted to 25 μL with TBS (pH 7.4) based on the dose, and then mixed with 25 μL of adjuvant 1. The mixture was stored in the absence of light to prevent oxidation.

### 2) Preparation of Control Vaccines

### Varilrix (Varicella Attenuated Vaccine):

This product contains a tubular injection vial made of borosilicate glass and a butyl rubber stopper (a pre-filled syringe containing 0.5 mL of a diluent per vial) and a vial (containing a lyophilized powder of the vaccine). The pre-filled syringe was connected to the vial, the diluent was completely injected into the vial to dissolve the lyophilized powder, the mixture was shaken for thorough mixing, and the vial should be clear and free of particulate matter after complete dissolution.

### Shingrix® (Recombinant Varicella-Zoster Vaccine):

After reconstitution, one dose (0.5 mL) contained 50 μg of gE protein. All AS01B adjuvant in the vial was extracted using a sterile disposable syringe to dissolve the lyophilized powder, the mixture was shaken for thorough mixing, and the vial should be clear and free of particulate matter after complete dissolution.

Shingrix 0.5 μg: Shingrix antigen protein was dissolved in 2500 μL of TBS, and the resulting solution was thoroughly mixed. Then, 250 μL of the solution was pipetted and thoroughly mixed with 250 μL of AS01B adjuvant. In the mouse model test, each dose (50 μL) contained 0.5 μg of antigen protein.

### (2) Immunization Procedures in Animal Experiment

a. Two Immunizations-Comparison of Linker Peptides 1 with Different Structures (VZV gE-NPM No Linker, VZV gE-NPM (G₄S)₃ Linker, and VZV gE-NPM (EAAAK)₃ Linker, where the (G₄S)₃ Linker and the (EAAAK)₃ Linker were Both Linker Peptides 1)

18 female BALB/c mice that had passed the acclimatization period were randomly divided into 3 groups, with 6 animals in each group. A total of 50 μL (25 μL per leg) was injected intramuscularly into the anterior or posterior portion of the tibial muscle of both legs of each mouse. Table 13 shows the experimental groups and the administration doses. The immunization procedures are as follows: primary immunization was performed on DO, secondary immunization was performed on D14, blood was collected on D28 after the primary immunization and the serum was isolated to determine the specific IgG antibody, and spleen was collected on D28 to isolate white blood cells for determination of cellular immunity indexes. The results are shown in panels d, e, and f in FIG. 1.

TABLE 13
Experimental groups and administration doses

| | Test sample/control | | | | | |
|---|---|---|---|---|---|---|
| | Immunogenic complex | | Adjuvant | | | |
| Group | | Dose | | Dose | Route of | Sampling |
| (n = 3) | Immunogenic complex | (μg) | Adjuvant | (μL) | administration | time |
| 1 | VZV gE-NPM | 2.5 | Adjuvant 1 | 25 | Intramuscular | Blood |
| | (no linker) | | | | injection on | sampling |
| 2 | VZV gE-NPM | 2.5 | Adjuvant 1 | 25 | D 0 and D 14 | and spleen |
| | Linker1 = (G₄S)₃; | | | | | collection |
| | SEQ ID NO: 16, | | | | | on D 28 |
| | SEQ ID NO: 18 | | | | | |
| 3 | VZV gE-NPM | 2.5 | Adjuvant 1 | 25 | | |
| | Linker1 = (EAAAK)₃, | | | | | |
| | SEQ ID NO: 15, | | | | | |
| | SEQ ID NO: 18) | | | | | |

b. Two Immunizations-Four Immunogenic Complexes and Shingrix (VZV gE-NPM, VZV gE-153-50, VZV gE-Ferritin, VZV gE-AP205, and Shingrix®)

40 female BALB/c mice that had passed the acclimatization period were randomly divided into 5 groups, with 8 animals in each group. A total of 50 μL (25 μL per leg) was injected intramuscularly into the anterior or posterior portion of the tibial muscle of both legs of each mouse. Table 14 shows the experimental groups and the administration doses. The immunization procedures are as follows: primary immunization was performed on DO, secondary immunization was performed on D14, blood was collected on D13 and D28 after the primary immunization and the serum was isolated to determine the specific IgG antibody, and spleen was collected on D28 to isolate white blood cells for determination of cellular immunity indexes.

In the experiment in this section (“b. Two immunizations-four immunogenic complexes and Shingrix®”), VZV gE-NPM was an immunogenic complex formed in Example 5 by the VZV gE-4T containing linker 1 (EAAAK)₃ (i.e., SEQ ID NO: 15) and NPM-4C set forth in SEQ ID NO: 18. The results are shown in FIG. 12.

TABLE 14
Experimental groups and administration doses

| | Test sample/control | | | | | |
|---|---|---|---|---|---|---|
| | Immunogenic complex | | Adjuvant | | | |
| Group | | Dose | | Dose | Route of | |
| (n = 5) | Immunogenic complex | (μg) | Adjuvant | (μL) | administration | Sampling time |
| 1 | VZV gE-NPM | 5 | Adjuvant 1 | 25 | Intramuscular | Blood sampling on |
| 2 | VZV gE-153-50 | 5 | Adjuvant 1 | 25 | injection on | D 13, blood sampling |
| 3 | VZV gE-Ferritin | 5 | Adjuvant 1 | 25 | D 0 and D 14 | and spleen collection |
| 4 | VZV gE-AP205 | 5 | Adjuvant 1 | 25 | | on D 28 |
| 5 | Shingrix ® | 5* | AS01B | 50 | | |

*The dose in the Shingrix ® group refers to the VZV gE protein

c. Primary Immunization with Attenuated Vaccine+Two Booster Immunizations

24 female BALB/c mice that had passed the acclimatization period were randomly divided into 3 groups, with 8 animals in each group. The administration volume was 50 μL/mouse. The immunization procedures are as follows: On D-35, 50 μL of Varilrix (varicella attenuated vaccine) was injected subcutaneously at the back of the neck.

The test samples were VZV gE-NPM (the dose of VZV gE-NPM was 0.5 μg and that of adjuvant 1 was 25 μL), Shingrix (the dose was 5 μg, and the dose of adjuvant AS01B was 50 μL), and Shingrix 0.5 (the dose was 0.5 μg, and the dose of adjuvant AS01B was 25 μL). The test samples were each administered once for immunization on Day 0 and Day 28 by intramuscular injection of a total of 50 μL (25 μL per leg) into the anterior or posterior portion of the tibial muscle of both legs. The day of the first immunization with the test sample was DO. Blood was collected on D14 and D58 and the serum was isolated to determine the specific IgG antibody, and spleen was collected on D58 to isolate white blood cells for determination of cellular immunity indexes. The results are shown in FIG. 13. VZV gE-NPM was an immunogenic complex formed by VZV gE-4T set forth in SEQ ID NO: 15 and NPM-4C set forth in SEQ ID NO: 18. The preparation method is shown in Example 5. The linker used for VZV gE-4T in FIG. 13 was (EAAAK)₃.

### (3) Detection of Specific IgG Antibody

The whole blood collected into the centrifuge tube was left to stand at room temperature for 2 h or left to stand overnight in a refrigerator at 4° C. After the blood was coagulated and the blood clot contracted, the blood was centrifuged at 4000 rpm for 10 min, and the supernatant was collected into a clean centrifuge tube and stored at −20° C.

A 96-well microplate (Thermo Fisher Scientific) was coated with VZV gE protein (at a concentration of 2 μg/mL) at 100 ng/50 μL/well and incubated at 4° C. overnight, and then the plate was washed twice with PBST (0.05% Tween 20). A blocking solution (Thermo Fisher Scientific) was added at 200 μL/well, followed by blocking at room temperature (25° C.±3° C.) for 1-4 h. Then, the plate was washed twice, diluted immune serum was added, and the mixture was incubated at room temperature for 1 h and then washed 4 times. A secondary antibody Goat Anti-Mouse IgG H&L (HRP) working solution diluted at 1:5000 was added at 50 μL/well. The plate was incubated at room temperature for 1 h and then washed 6 times, and a chromogenic solution was added at 100 μL/well. Color development was performed at room temperature for 10 min in the absence of light, and then 1 M HCL was added at 100 μL/well to stop the reaction. The main wavelength was set at 450 nm and the reference wavelength was set at 620 nm in the microplate reader. The absorbance value of the sample=OD450−OD620. The measurement was completed within 5 min after the termination.

### Data Processing:

The following conditions were met and the data were reliable:

The OD value of the control serum ±0.2, the OD value corresponding to the initial concentration of the sample was <3.0, the OD value corresponding to the blank well was less than 0.1, and the coefficient of variation of duplicate wells (response values) should be less than 20%.

The original sample data was applied to “excel Endpoint ELISA template” to determine the antibody titer. In calculation, the cut off value was 0.15. Geometric mean titers (GMT) were presented in bar graphs.

### (4) Mouse Cytokine ELISpot Assay

After blood collection and euthanasia of the mice, sterile operations were performed in an ultra-clean bench. The mouse was immobilized, the abdominal cavity was exposed, and the spleen was excised. Forceps were used to place the spleen into a sample tube containing an appropriate amount of pre-chilled and sterile 1×PBS, ensuring the spleen was completely immersed. The spleen was dissociated into single cells using a tissue dissociator (Miltenyi). The cells were then added to a 96-well assay plate from a kit, which had been washed 4 times with PBS and pre-conditioned with α-MEM complete medium for 1-4 h.

The cells were set at 3 densities, i.e., 5×10⁶ cells/50 μL/well, 2.5×10⁶ cells/50 μL/well, and 1.25×10⁶ cells/50 μL/well, and then the polypeptide stimulator was added at 100 ng/50 μL/well. The negative control wells (set for each animal) and the positive control wells contained 5×10⁶ cells/50 μL/well, and the negative control wells were not stimulated with the polypeptide and were supplemented with 50 μL of complete medium. 50 μL of the positive stimulator (the final concentration of PMA was 6 μg/mL and the final concentration of ION was 2 μg/mL) was added to the positive control wells. The cells were cultured in an incubator at 37° C. with 5% CO₂ for about 20 h. The subsequent steps were performed according to the kit (MABTECH) instructions. The biotinylated monoclonal antibody, Streptavidin-ALP, and the chromogenic substrate BCIP/NBT-plus were added sequentially, and after 10 min, the plate was slowly washed with tap water to stop the chromogenic reaction. The reaction strip was air-dried at room temperature in the absence of light, and the spots were counted using an ELISpot reader.

### Data Processing:

$$Number of cytokine spots = number of spots in polypeptide stimulation wells ( cell density 5 \times {10}^{6} cells / 50 µL / well ) - number of spots in self - negative control wells ( cell density 5 \times {10}^{6} cells / 50 µL / well )$$

Mean values were presented in bar graphs.

### (5) Statistical Analysis

The results were analyzed using Graphpad Prism 9.1.2 software. Differences were analyzed using Unpaired t test or One-Way ANOVA. Two groups of data were defined as having a significant difference when P<0.05.

### 3. Test Results:

(1) as can be Seen from Results Obtained with “a. Two Immunizations” Shown in Panels d, e, and f in FIG. 1:

FIG. 1d showed IFN-γ levels on day 28, FIG. 1e showed IL-2 levels on day 28, and FIG. if showed IgG levels on day 28. For the linker peptide 1 (linker1), the molecular designs incorporating a linker (whether (G₄S)₃ or (EAAAK)₃) elicited stronger immunogenicity than the design without linker 1 did. This was demonstrated by significantly higher cellular immune responses induced by the linker 1-containing designs than that induced by linker 1-free design, along with a trend toward higher antibody responses. Meanwhile, it was found that using (EAAAK)₃ as linker1 can induce significantly stronger immune responses than using (G₄S)₃.

As can be seen from results obtained with “b. two immunizations” shown in FIG. 12: for antibody responses: as shown in FIG. 12, panel a showed the comprehensive evaluation results of four nanoparticle groups on D13 (day 13), which showed that the effects were all higher than those of the control group, where the VZV gE-NPM, VZV gE-I53-50, and VZV gE-Ferritin nanoparticle systems all produced significantly higher antibody titers than that of the control group, while the VZV gE-AP205 group also showed a trend of higher antibody titer than that of the control group. Panel b showed that all nanoparticle groups exhibited significant differences in D28 (day 28) antibody titers as compared to the control group.

Cell response: Panels c and d showed the spleen cytokine assay results on D28 (day 28), and the results showed that VZV gE-NPM induced significantly higher IFN-γ and IL-2 responses than the control group did. The VZV gE-I53-50 and VZV gE-Ferritin groups induced significantly higher IL-2 response than the control group did. Overall, the cytokine responses of all nanoparticle groups were higher than that of the control vaccine.

It can be seen that when a non-potent adjuvant is used, the nanoparticle platform disclosed herein (four immunogenic complexes: VZV gE-NPM, VZV gE-I53-50, VZV gE-Fe, and VZV gE-AP205) can induce cellular and humoral immune responses better than that of Shringrix, and meanwhile, in terms of side effects or safety, the combination of the varicella-zoster vaccines prepared using the nanoparticle platform with the non-potent adjuvant exhibits significant advantages.

(2) as can be Seen from Results Obtained with “c. Primary Immunization with Attenuated Vaccine+Two Booster Immunizations” Shown in FIG. 13:

After two sequential booster immunizations of the mice following primary immunization with attenuated varicella vaccine (mimicking infection), VZV gE-NPM induced excellent humoral immune response and induced IFN-γ and IL-2 cytokine immune responses significantly higher than those of the control group at the same antigen dose. In addition, as can be seen from FIG. 13, a 1/10 dose of VZV gE-NPM was sufficient to achieve efficacy comparable to that of the full 5 μg dose of Shingrix. It is demonstrated that the particle vaccines are superior to the control vaccine under different immunization procedures.

### Example 11: Immunogenicity Test of VZV gE-NPM in Combination with Adjuvants with Different Squalene Contents in Balb/c Mice

### I. Reference was Made to Example 10 for Experimental Materials and Experimental Method.

1. The squalene content in the adjuvants used in different experimental groups was adjusted (the corresponding content of the remaining components in the adjuvants was also adaptively adjusted, specifically as follows, but the change of the remaining components did not affect the immune effect).

The contents of the components in squalene-based adjuvants used in the different groups are as follows:

Adjuvant 25 μL group (group 1): the squalene content was 4.03% (w/w), equivalent to 1.01 mg, i.e., 40.3 mg/mL; the Span 85 content was 0.5% (w/w), equivalent to 0.125 mg, i.e., 5 mg/mL; the Tween 80 content was 0.5% (w/w), equivalent to 0.125 mg, i.e., 5 mg/mL; the citric acid content was 0.016% (w/w), equivalent to 0.004 mg, i.e., 0.16 mg/mL; the sodium citrate content was 0.264% (w/w), equivalent to 0.066 mg, i.e., 2.64 mg/mL.

Adjuvant 2.5 μL group (group 2): the squalene content was 0.403% (w/w), equivalent to 0.01 of the mass of the squalene in group 1, equivalent to 0.101 mg, i.e., 4.03 mg/mL; the Span 85 content was 0.05% (w/w), equivalent to 0.01 of the mass of the Span in group 1, equivalent to 0.0125 mg, i.e., 0.5 mg/mL; the Tween 80 content was 0.05% (w/w), equivalent to 0.01 of the mass of the Tween 80 in group 1, equivalent to 0.0125 mg, i.e., 0.5 mg/mL; the citric acid content was 0.0016% (w/w), equivalent to 0.01 of the mass of the citric acid in group 1, equivalent to 0.0004 mg, i.e., 0.016 mg/mL; the sodium citrate content was 0.0264% (w/w), equivalent to 0.01 of the mass of the sodium citrate in group 1, equivalent to 0.0066 mg, i.e., 0.264 mg/mL.

Adjuvant 18.75 μL group (group 3): the squalene content was 3.0225% (w/w), equivalent to 0.75 of the mass of the squalene in group 1, equivalent to 0.7575 mg, i.e., 30.225 mg/mL; the Span 85 content was 0.375% (w/w), equivalent to 0.75 of the mass of the Span in group 1, equivalent to 0.0938 mg, i.e., 3.75 mg/mL; the Tween 80 content was 0.375% (w/w), equivalent to 0.75 of the mass of the Tween 80 in group 1, equivalent to 0.0938 mg, i.e., 3.75 mg/mL; the citric acid content was 0.012% (w/w), equivalent to 0.75 of the mass of the citric acid in group 1, equivalent to 0.003 mg, i.e., 0.12 mg/mL; the sodium citrate content was 0.198% (w/w), equivalent to 0.75 of the mass of the sodium citrate in group 1, equivalent to 0.0795 mg, i.e., 1.98 mg/mL.

Adjuvant 12.5 μL group (group 4): the squalene content was 2.015% (w/w), equivalent to 0.5 times the mass of the squalene in group 1, i.e., 0.505 mg, i.e., 20.15 mg/mL; the Span 85 content was 0.25% (w/w), equivalent to 0.5 times the mass of the Span 85 in group 1, equivalent to 0.0625 mg, i.e., 2.5 mg/mL; the Tween 80 content was 0.25% (w/w), equivalent to 0.5 times the mass of the Tween 80 in group 1, equivalent to 0.0625 mg, i.e., 2.5 mg/mL; the citric acid content was 0.08% (w/w), equivalent to 0.5 of the mass of the citric acid in group 1, equivalent to 0.002 mg, i.e., 0.08 mg/mL; the sodium citrate content was 0.132% (w/w), equivalent to 0.5 of the mass of the sodium citrate in group 1, equivalent to 0.033 mg, i.e., 1.32 mg/mL.

Control group (NA): containing VZV gE-NPM only, no adjuvant.

2. The VZV gE-NPM antigen protein was administered at a dose of 5 μg to the animal model in each group. The primary immunization was performed on Day 0, followed by an intermediate blood collection and a booster immunization on day 14. On day 28, whole blood was taken to isolate serum, and the spleen was collected. VZV gE-NPM was an immunogenic complex formed by VZV gE-4T set forth in SEQ ID NO: 15 and NPM-4C set forth in SEQ ID NO: 18. The preparation method is shown in Example 5.

### II. Experimental Results

As shown in FIG. 14, the VZV gE-NPM samples in this experiment were used for basic immunizations on days 0 and 14, and the dose administered to the animal model was 5 μg. Sampling was performed on day 28 for detection and analysis. Data were analyzed by common one-way ANOVA with Dunnett's multiple comparison test, and the results are presented in the graphs.

Under the condition of the same antigen dose (5 μg gE-NPM protein), the application of adjuvants with different squalene contents revealed that both the 18.75 μL and 12.5 μL groups achieved efficacy comparable to that of the 25 μL group, and the 18.75 μL group demonstrated even superior efficacy than that of the 25 μL group. Since the dose of the VZV gE-NPM antigen protein used for animal model in each group was 5 μg, the mass ratios of VZV gE-NPM to squalene were known, i.e., 202:1 (group 1), 20.2:1 (group 2), 151.5:1 (group 3), and 101:1 (group 4).

It can be seen from the grading results from sampling on day 28 that in the present disclosure, the vaccines composed of VZV gE-NPM as a representative component can also exert ideal immune effects under the conditions of adjuvants with different degrees of low squalene content.

In the present disclosure, the squalene-based adjuvant in the recombinant herpes zoster vaccine for human use preferably comprises, per unit dose, 10.50 mg (4.2%) of squalene, 1.25 mg (0.5%) of Span 85, 1.25 mg (0.5%) of Tween 80, 0.04 mg (0.264%) of citric acid, and 0.66 mg (0.016%) of sodium citrate (w/w).

### Example 12: Stability Examination of Different Composition Formulas of VZV gE-NPM Vaccine Lyophilized Formulations and Screening and Determination of Optimal Lyophilization Formula

1. Experimental method: Different formula compositions were designed, and formulas composed of different amounts of alcohol, amino acid, and surfactant were selected. The purity of the vaccine compositions was measured by SEC-HPLC and SDS-PAGE methods, and the stability of the different formulas was compared.

The VZV gE-NPM protein in the formula was 0.1 mg/mL, and the high temperature means placing the lyophilized product in a 40° C. thermostatic incubator. Physicochemical assay (SDS-PAGE and SEC-HPLC) was performed on day 3, day 7, day 14, and day 28.

2. Experimental results: The results are shown in Table 15 below.

As can be seen from the above experimental results, the influence of various factors on the stability of the particle protein vaccine disclosed herein was different. The content of the immunogenic complex was kept unchanged (VZV gE-NPM, 0.1 mg/mL), the optimal types and concentrations of the saccharide, the alcohol, the amino acid, and the surfactant were determined by screening as follows: sucrose, 25 mg/mL; mannitol, 50 mg/mL; arginine, 8.7 mg/mL; and Tween 80, 0.5 mg/mL. VZV gE-NPM was an immunogenic complex formed by VZV gE-4T set forth in SEQ ID NO: 15 and NPM-4C set forth in SEQ ID NO: 18. The preparation method thereof is shown in Example 5.

Thus, the formula of the VZV gE-NPM lyophilized formulation was determined, and the formula comprises: 25 μg or 50 μg of VZV gE-NPM, 12.5 mg of sucrose, 25 mg of mannitol, 0.25 mg of Tween 80, 4.35 mg of arginine, 1.085 mg of disodium hydrogen phosphate dihydrate, 0.62 mg of sodium dihydrogen phosphate dihydrate, and 8.66 mg of hydrochloric acid.

The key temperatures of the above optimal formula for the lyophilized formulation were determined through experiments: a collapse temperature Tc of −39° C., a glass transition temperature Tg of 51.6° C., and a glass transition temperature Tg′ of −55° C. (Tc, Tg, and Tg′ are the key temperatures for lyophilized formulations and are used for guiding the setting of the pre-freezing and primary drying temperatures during the lyophilization process, as well as the maximum storage temperature. Typically, the pre-freezing temperature is lower than Tg′, the primary drying temperature is lower than Tc, and the storage temperature of the finished product is lower than Tg).

Considering the extreme conditions that the lyophilized vaccine formulations may be subjected to during transportation and storage, the lyophilized formulation based on the above formula was subjected to high temperature (40° C. 7 d and 14 d), vibration (the sample was fixed on a decolonization shaker, with the rotation speed set to 240 rpm, and the duration set to 24 h), and illumination (the sample was placed vertically in a 4° C. light box for 3 d). 24 h after reconstitution, the in vivo potency assay showed that the activity of the formulation samples after the above treatment was stable (female BALB/c mice aged 5-6 weeks were used, subjected to primary immunization on D0, secondary immunization on D14, intermediate blood collection on D14, and end-point blood collection and spleen collection on D28). The immunological activity was well maintained.

Therefore, the formula composition of the recombinant herpes zoster vaccine (lyophilized formulation) for human use per unit dose was finally determined. The vaccine was prepared by reconstituting the VZV gE-NPM lyophilized formulation for injection with the adjuvant (0.5 mL/dose). The formula of the lyophilized formulation is shown in Table 16 below.

The amount of VZV gE-NPM and adjuvant used is different for human vaccination and mouse vaccination. When used in humans, the amount of VZV gE-NPM and that of the adjuvant were both 10 times the amount used in mice, e.g., 5 μg/dose of VZV gE-NPM for mice versus 50 μg/dose for humans, or 50 μL/dose of adjuvant for mice versus 500 μL/dose (0.5 mL/dose) of adjuvant for humans.

TABLE 15
Formula screening for lyophilized formulation of recombinant herpes zoster vaccine VZV gE-NPM

| Saccharide | | Alcohol | | Amino acid | | Surfactant | | |
|---|---|---|---|---|---|---|---|---|
| | Concentration | | Concentration | | Concentration | | Concentration | |
| Type | (mg/mL) | Type | (mg/mL) | Type | (mg/mL) | Type | (mg/mL) | Results |
| Sucrose | 25 | Mannitol | 50 | Arginine | 17.4 | Tween | 0.5 | Appearance: white intact |
| | | | | | | 20 | | lyophilized cake, |
| | | | | | | | | aggregates observed by |
| | | | | | | | | physicochemical assay |
| | | | | | | | | after being left to stand |
| | | | | | | | | at high temperature |
| Sucrose | 25 | Mannitol | 50 | Arginine | 8.7 | Tween | 0.5 | Appearance: white intact |
| | | | | | | 80 | | lyophilized cake, stable |
| | | | | | | | | in physicochemical assay |
| | | | | | | | | after being left to stand |
| | | | | | | | | at high temperature for 1 |
| | | | | | | | | month |
| Sucrose | 25 | Mannitol | 50 | Arginine | 3.48 | Tween | 0.5 | Appearance: white intact |
| | | | | | | 80 | | lyophilized cake, |
| | | | | | | | | aggregates observed by |
| | | | | | | | | physicochemical assay |
| | | | | | | | | after being left to stand |
| | | | | | | | | at high temperature |
| Sucrose | 25 | Mannitol | 50 | Arginine | 0.87 | Tween | 0.5 | Appearance: white intact |
| | | | | | | 80 | | lyophilized cake, |
| | | | | | | | | aggregates observed by |
| | | | | | | | | physicochemical assay |
| | | | | | | | | after being left to stand |
| | | | | | | | | at high temperature |
| Sucrose | 25 | Mannitol | 50 | Histidine | 7.75 | Tween | 0.5 | Appearance: white intact |
| | | | | | | 80 | | lyophilized cake, a small |
| | | | | | | | | amount of aggregates |
| | | | | | | | | observed by |
| | | | | | | | | physicochemical assay |
| | | | | | | | | after being left to stand |
| | | | | | | | | at high temperature |
| Sucrose | 25 | Mannitol | 50 | Histidine | 3.1 | Tween | 0.5 | Appearance: white intact |
| | | | | | | 80 | | lyophilized cake, |
| | | | | | | | | aggregates observed by |
| | | | | | | | | physicochemical assay |
| | | | | | | | | after being left to stand |
| | | | | | | | | at high temperature |
| Sucrose | 25 | Mannitol | 50 | Histidine | 1.55 | Tween | 0.5 | Appearance: white intact |
| | | | | | | 80 | | lyophilized cake, |
| | | | | | | | | aggregates observed by |
| | | | | | | | | physicochemical assay |
| | | | | | | | | after being left to stand |
| | | | | | | | | at high temperature |
| Sucrose | 25 | Mannitol | 50 | Histidine | 0.775 | Tween | 0.5 | Appearance: white intact |
| | | | | | | 80 | | lyophilized cake, |
| | | | | | | | | aggregates observed by |
| | | | | | | | | physicochemical assay |
| | | | | | | | | after being left to stand |
| | | | | | | | | at high temperature |

TABLE 16
Formula composition of lyophilized
formulation of VZV gE-NPM vaccine

| | Formula | | Concentration of each | |
|---|---|---|---|---|
| Ingredient | content | | component in formula | |
| VZV-gEM protein | 50 μg or 25 μg | | 0.1 mg/mL or 0.05 mg/mL | |
| Sucrose | 12.5 | mg | 25 | mg/mL |
| Mannitol | 25 | mg | 50 | mg/mL |
| Tween 80 | 0.25 | mg | 0.5 | mg/mL |
| Arginine | 4.35 | mg | 8.7 | mg/mL |
| Disodium hydrogen | 1.085 | mg | 2.17 | mg/mL |
| phosphate dihydrate | | | | |
| Sodium dihydrogen | 0.62 | mg | 1.24 | mg/mL |
| phosphate dihydrate | | | | |

| | | | |
|---|---|---|---|
| Hydrochloric acid | 8.66 | mg | pH regulator |

In summary, the above examples and drawings are only for the purpose of illustrating preferred examples disclosed herein, and are not intended to limit the protection scope of the present disclosure. Any modifications, equivalent substitutions, improvements, and the like made without departing from the spirit and principle of the present disclosure shall all fall in the protection scope of the present disclosure.

1.-10. (canceled)

11. An immunogenic complex, comprising:
an antigenic component consisting of a varicella zoster virus (VZV) gE protein or an immunogenic fragment thereof, a linker peptide 1, and a binding peptide 1; and
a particle protein component consisting of a nanoparticle protein, a linker peptide 2, and a binding peptide 2;
wherein the antigenic component and the particle protein component are covalently bound to each other via the binding peptide 1 and the binding peptide 2.

12. The immunogenic complex according to claim 11, wherein the immunogenic complex is characterized by any one or more of (1)-(6) below:
(1) the amino acid sequence of the varicella zoster virus (VZV) gE protein is set forth in SEQ ID NO: 14;
(2) the amino acid sequence of the linker peptide 1 is set forth in SEQ ID NO: 3 or SEQ ID NO: 4;
(3) the amino acid sequence of the binding peptide 1 is set forth in SEQ ID NO: 1;
(4) the nanoparticle protein is selected from NPM, AP205, or Ferritin, wherein the amino acid sequence of the NPM is set forth in SEQ ID NO: 17, the amino acid sequence of the Ferritin is set forth in SEQ ID NO: 22, and the amino acid sequence of the AP205 is set forth in SEQ ID NO: 24;
(5) the amino acid sequence of the linker peptide 2 is set forth in SEQ ID NO: 7, SEQ ID NO: 8, or SEQ ID NO: 9; and
(6) the amino acid sequence of the binding peptide 2 is set forth in SEQ ID NO: 2.

13. The immunogenic complex according to claim 11, wherein the varicella zoster virus (VZV) gE protein is expressed using a signal peptide with the amino acid sequence set forth in any one of SEQ ID NOs: 10-12, and the antigenic component and/or the particle protein component comprise a histidine tag.

14. The immunogenic complex according to claim 12, wherein the varicella zoster virus (VZV) gE protein is expressed using a signal peptide with the amino acid sequence set forth in any one of SEQ ID NOs: 10-12, and the antigenic component and/or the particle protein component comprise a histidine tag.

15. The immunogenic complex according to claim 13, wherein the amino acid sequence of the antigenic component is set forth in SEQ ID NO: 15, and the amino acid sequence of the particle protein component is set forth in SEQ ID NO: 18.

16. The immunogenic complex according to claim 14, wherein the amino acid sequence of the antigenic component is set forth in SEQ ID NO: 15, and the amino acid sequence of the particle protein component is set forth in SEQ ID NO: 18.

17. A preparation method for the immunogenic complex according to claim 11, comprising:
ligating an encoding gene of the antigenic component and an encoding gene of the particle protein component into expression vectors respectively to construct recombinant expression plasmids and expression host strains, and then expressing and purifying target proteins; and
co-incubating the antigenic component and the particle protein component obtained in step (1) to obtain the immunogenic complex.

18. An immune composition, comprising the immunogenic complex according to claim 11 and a pharmaceutically acceptable carrier, wherein the pharmaceutically acceptable carrier comprises a stabilizer, an excipient, a surfactant, a buffering agent, and a pH regulator, wherein the stabilizer is sucrose and arginine, the excipient is mannitol, the surfactant is Tween 80, the buffering agent is disodium hydrogen phosphate dihydrate and sodium dihydrogen phosphate dihydrate, and the pH regulator is hydrochloric acid.

19. An immune composition, comprising the immunogenic complex according to claim 12 and a pharmaceutically acceptable carrier, wherein the pharmaceutically acceptable carrier comprises a stabilizer, an excipient, a surfactant, a buffering agent, and a pH regulator, wherein the stabilizer is sucrose and arginine, the excipient is mannitol, the surfactant is Tween 80, the buffering agent is disodium hydrogen phosphate dihydrate and sodium dihydrogen phosphate dihydrate, and the pH regulator is hydrochloric acid.

20. An immune composition, comprising the immunogenic complex according to claim 13 and a pharmaceutically acceptable carrier, wherein the pharmaceutically acceptable carrier comprises a stabilizer, an excipient, a surfactant, a buffering agent, and a pH regulator, wherein the stabilizer is sucrose and arginine, the excipient is mannitol, the surfactant is Tween 80, the buffering agent is disodium hydrogen phosphate dihydrate and sodium dihydrogen phosphate dihydrate, and the pH regulator is hydrochloric acid.

21. An immune composition, comprising the immunogenic complex according to claim 14 and a pharmaceutically acceptable carrier, wherein the pharmaceutically acceptable carrier comprises a stabilizer, an excipient, a surfactant, a buffering agent, and a pH regulator, wherein the stabilizer is sucrose and arginine, the excipient is mannitol, the surfactant is Tween 80, the buffering agent is disodium hydrogen phosphate dihydrate and sodium dihydrogen phosphate dihydrate, and the pH regulator is hydrochloric acid.

22. The immune composition according to claim 18, wherein the immune composition is a lyophilized formulation comprising, per unit dose, 25-50 μg of VZV gE-NPM, 12-15 mg of sucrose, 20-25 mg of mannitol, 3-5 mg of arginine, 0.2-0.3 mg of polysorbate 80, 1-1.2 mg of disodium hydrogen phosphate dihydrate, 0.6-0.8 mg of sodium dihydrogen phosphate dihydrate, and 8.0-9.5 mg of hydrochloric acid.

23. The immune composition according to claim 19, wherein the immune composition is a lyophilized formulation comprising, per unit dose, 25-50 μg of VZV gE-NPM, 12-15 mg of sucrose, 20-25 mg of mannitol, 3-5 mg of arginine, 0.2-0.3 mg of polysorbate 80, 1-1.2 mg of disodium hydrogen phosphate dihydrate, 0.6-0.8 mg of sodium dihydrogen phosphate dihydrate, and 8.0-9.5 mg of hydrochloric acid.

24. The immune composition according to claim 20, wherein the immune composition is a lyophilized formulation comprising, per unit dose, 25-50 μg of VZV gE-NPM, 12-15 mg of sucrose, 20-25 mg of mannitol, 3-5 mg of arginine, 0.2-0.3 mg of polysorbate 80, 1-1.2 mg of disodium hydrogen phosphate dihydrate, 0.6-0.8 mg of sodium dihydrogen phosphate dihydrate, and 8.0-9.5 mg of hydrochloric acid.

25. The immune composition according to claim 21, wherein the immune composition is a lyophilized formulation comprising, per unit dose, 25-50 μg of VZV gE-NPM, 12-15 mg of sucrose, 20-25 mg of mannitol, 3-5 mg of arginine, 0.2-0.3 mg of polysorbate 80, 1-1.2 mg of disodium hydrogen phosphate dihydrate, 0.6-0.8 mg of sodium dihydrogen phosphate dihydrate, and 8.0-9.5 mg of hydrochloric acid.

26. A varicella zoster vaccine, comprising the immune composition according to claim 22 and an adjuvant, wherein the adjuvant comprises (w/w) 1.5%-5% squalene, 0.05%-1% Span 85, 0.05%-1% Tween 80, and 10 mM citrate buffer.

27. A varicella zoster vaccine, comprising the immune composition according to claim 23 and an adjuvant, wherein the adjuvant comprises (w/w) 1.5%-5% squalene, 0.05%-1% Span 85, 0.05%-1% Tween 80, and 10 mM citrate buffer.

28. A varicella zoster vaccine, comprising the immune composition according to claim 24 and an adjuvant, wherein the adjuvant comprises (w/w) 1.5%-5% squalene, 0.05%-1% Span 85, 0.05%-1% Tween 80, and 10 mM citrate buffer.

29. A varicella zoster vaccine, comprising the immune composition according to claim 25 and an adjuvant, wherein the adjuvant comprises (w/w) 1.5%-5% squalene, 0.05%-1% Span 85, 0.05%-1% Tween 80, and 10 mM citrate buffer.

30. A method of preventing or treating herpes zoster comprising the use of the immunogenic complex according to claim 11.
